(19) United States
(12) Patent Application Publication
(10) Pub. No.: US 2003/0009145 A1
(43) Pub. Date: Jan. 9, 2003

(54) Delivery of drugs from sustained release devices implanted in myocardial tissue or in the pericardial space

(76) Inventors: Harry A.J. Struijker-Boudier, Rijckholt (NL); Johannes J.R. Hermans, Schimmert-Nuth (NL); Jos F.M. Smits, Eijsden (NL); Randolph M. Johnson, Half Moon Bay, CA (US); Felix Theeuwes, Los Altos (BE)

Correspondence Address: DURECT CORPORATION, 10240 BUBB ROAD, CUPERTINO, CA, 95014, US

(21) Appl. No.: 10/104,247

(22) Filed: Mar. 22, 2002

Related U.S. Application Data
(60) Provisional application No. 60/278,518, filed on Mar. 23, 2001.
(60) Provisional application No. 60/311,309, filed on Aug. 9, 2001.
(60) Provisional application No. 60/347,326, filed on Jan. 9, 2002.

(51) Int. Cl. A61M 31/00

(52) U.S. Cl. 604/500; 604/891.1

(57) ABSTRACT

The present invention provides delivery of drugs to the heart or cardiac vasculature using fully implanted sustained-release dosage forms.

This application claims priority from U.S. patent application Ser. No. 60/278,518 filed Mar. 23, 2001, U.S. patent application Ser. No. 60/311,309 filed Aug. 9, 2001, and U.S. patent application Ser. No. 60/347,326 filed Jan. 9, 2002.

### FIELD OF THE INVENTION

This invention is in the field of sustained-release drug delivery to the heart, specifically to implanted, sustained-release drug delivery dosage forms implanted in the heart tissue or in the pericardial space, or sprayed directly onto the surface of the heart.

### BACKGROUND OF THE INVENTION

Anatomy of the Heart

The heart is surrounded by the pericardium, which is a sac consisting of two layers of tissue (fibrous pericardium and parietal layer of the serous pericardium). The pericardial space, between the pericardium and the heart, contains some pericardial fluid that bathes the outer tissue heart in a stable osmotic and electrolytic environment. The heart tissue itself consists of four layers, the visceral layer of the serous pericardium, an adipose layer containing embedded arteries and veins, the myocardium, which is the major, muscular layer of the heart, and the inner epithelial layer, called the endocardium (“Cardiopulmonary anatomy and physiology” Hicks; W. B. Saunders 2000).

The coronary arteries are the first vessels to branch off the aorta. Through these arteries, the heart receives (at rest) about 5% of the cardiac output. Coronary blood flow is governed by a pressure gradient and by resistance of the vessels.

Ischemic Disease of the Heart and Traditional Treatment

Coronary blood flow may be seriously reduced in coronary artery disease, and, as a result, the myocardium may become ischemic (starved of oxygen) or even infarcted (necrotic). The most common cause of myocardial ischemia is coronary atherosclerosis, which produces progressive stenosis (narrowing of the lumen), reducing coronary blood flow. The atherosclerotic plaque (consisting of cholesterol, lipids and cellular debris) causes progressive obstruction of the lumen and generates a high resistance area. The pressure drop will be higher than normal in this segment, and the perfusion pressure will be lower at the point distal to the obstruction. In this regard, collateral circulation is important, because if obstruction is total, myocardial infarction is likely to occur, particularly if the heart does not find a compensatory mechanism to supply the suffering myocardium. In this situation, the body will attempt to increase coronary blood flow, but the narrowed segment will offer great resistance and regional ischemia will develop if compensatory mechanisms fail, leading to heart attack.

Occlusive vascular disease (e.g. coronary artery disease) may be treated using a number of clinical techniques including angioplasty. Angioplasty is a procedure in which a balloon is inserted into the vessel and then inflated to dilate the area of narrowing. During inflation, the balloon can damage the vessel wall. It appears that as a result of this damage, in 30 to 50% of cases, the initial increase in lumen dimensions is followed by a localized re-narrowing (restenosis) of the vessel over a time of three to six months. Thus, restenosis can result in the dangerous and localized re-narrowing of a patient's vessel at the site of the recent angioplasty. Often, the only practical option is to perform repeated angioplasty, with its inherent risks, expense and shortcomings. Gibbons et al., Molecular Therapies for Vascular Diseases, Science vol. 272, pages 617-780 (May 1996).

Restenosis, like many other localized injuries and diseases, has responded poorly to pharmacological therapies and agents. Numerous pharmacological agents have been clinically tested, including anti-proliferatives such as rapamycin, taxol and taxol derivatives, which have shown some recent success. But it has been suggested that even better results may be possible if anti-restenosis drugs could be delivered at higher concentrations to the local site of intended action. In present therapies, anti-restenosis drugs may be delivered at sub-optimal concentrations locally, because to achieve optimal local dosing, the systemic dose required would produce serious side-effects. For example, taxol is an anti-mitotic drug that disrupts microtubule formation, and may well have pleiotropic undesired effects, for instance on bone-marrow stem cells and other highly mitotic cell populations.

Currently used systems for localized delivery of drugs to a treatment site inside a blood vessel includes use of dual balloon delivery systems that have proximal and distal balloons that are simultaneously inflated to isolate a treatment space within an arterial lumen. A catheter extends between the two balloons to locally deliver a therapeutic agent. Other balloon-based localized delivery systems include porous balloon systems, hydrogel-coated balloons and porous balloons that have an interior metallic stent. Other systems include locally placed drug-loaded coated metallic stents and drug-filled polymer stents. Wilensky et al., Methods and Devices for Local Drug Delivery in Coronary and Peripheral Arteries, Trend Cardiovasc Med, vol. 3 (1993).

These balloon devices provide far from ideal treatment, and their efficacy is limited by a number of factors including the rate of fluid flux through the vascular wall, the residence time of the deposited agent and the local conditions and vasculature of the deposition site. Further, to the extent that these systems allow the therapeutic agent to be carried away, these systems run the risk of applying a therapeutic agent to areas of the patient's vasculature where such agents may not be beneficial.

Angiogenic Factors

An experimental approach to the treatment of occlusive vascular disease (e.g. coronary artery disease) is to encourage the growth of new blood vessels that would replenish the blood supply to ischemic tissue using angiogenic factors. A major problem with delivery of such drugs is that of appropriate and effective local delivery.

Various angiogenic factors are known that promote the growth of blood vessels, e.g., Vascular Endothelial Growth Factor (VEGF), FGF, platelet derived growth factor, endothelial mitogenic growth factor etc.

Methods and Devices for Drug Delivery

Controlled release drug delivery for epicardial or endocardial therapies have been described variously over the years. In an epicardial therapy, it was first described by Folkman and Long in 1964 (“Drug Pacemakers in the Treatment of Heart Block”, New York Acad. Sci., Jun. 11, 1964, p. 857). They described a wax or silicone rubber capsule technology capable of being loaded with candidate cardiac active agents. In open chest animal studies, a capsule was tunneled into the epicardial tissue. After being thus positioned, the capsule released its agent producing quantifiable effects on heart rate for four to five days. After this period of time, increased heart rate gradually returned to normal. In 1983, Stokes, et al. (“Drug Eluting Electrodes. Improved Pacemaker Performance”, IEEE Trans. Biomed. Eng., Vol. BME-29, 1982, p. 614), described a steroid endocardial pacing electrode for purposes of reducing pacing thresholds. In 1987, Stokes, et al. (“Epicardial Lead Having Low Threshold, Low Polarization Myocardial Electrode”, US H356, Nov. 3, 1987) described a myocardial pacing electrode with drug delivery capabilities. Although not specifically described, myocardial electrodes generally require a transchest surgical procedure in order to screw or in some fashion, impale the electrode into the heart tissue.

Beginning in 1987, Levy's group at the University of Michigan (U.S. Pat. No. 5,387,419; PCT Appl. US 94/02838; and “Drug Delivery Polyurethane as Myocardial Implant for Antiarrhythmic Therapy”, Proc. Intern. Symp. Cont. Rel. Bioact. Mat., Vol. 14, 1987, p. 257) described the acute effects of an epicardially positioned, polymeric drug loaded patch in induced ventricular tachycardia (VT) in open chest animal models. These studies showed the ability of these systems to convert induced VT to normal single rhythm (NSR) in the animal model. In 1994, Labhasetwar, et al. (“Epicardial Administration of Ibutilide rom Polyurethane Matrices: Effects on Defibrillation Threshold and Electrophysiologic Parameters”, J. Cardiovasc. Pharm., Vol. 24, 1994, pp. 826-840), first described the reduction of defibrillation thresholds using epicardially positioned patch containing ibutilide in the acute canine model. In 1992, Moaddeb (U.S. Pat. No. 5,154,182) described an implantable, patch electrode, capable of delivering a drug, which is “ . . . surgically attached . . . ” to the epicardium. Such devices might be able to release a candidate substance into the epicardial space for purposes such as reducing defibrillation threshold, and reducing inflammation.

Various other methods and devices have been developed for delivering therapeutic agents to cardiac tissue. For example, U.S. Pat. Nos. 5,387,419; 5,931,810; 5,827,216; 5,900,433; 5,681,278; and 5,634,895 and PCT Publication No. WO 97/16170 discusses various devices and/or methods of delivering agents to the heart by, for example, transpericardial delivery.

U.S. Pat. Nos. 5,387,419 and 5,797,870 discuss methods for delivery of agents to the heart by admixing the agent with a material to facilitate sustained or controlled release of agent from a device, or by admixing the agent with a viscosity enhancer to maintain prolonged, high pericellular agent concentration.

Other proposed methods for site-specific delivery of drugs include the direct deposition of therapeutic agents into the arterial wall, systemic administration of therapeutic agents that have a specific affinity for the injured or diseased tissue, and systemic administration of inactive agents followed by local activation. For example, U.S. Pat. No. 6,251,418 discloses a method for implanting solid polymer pellets into myocardial tissue, where the pellets are coated with or contain a drug.

U.S. Pat. No. 6,258,119 describes a myocardial implant for insertion into a heart wall for trans myocardial revascularization (TMR) of the heart wall. The implant provides a means to promote angiogenesis, and has a flexible, elongated body that contains a cavity and openings through the flexible, elongated body from the cavity. The TMR implant includes a coaxial anchoring element integrally formed at one end for securing the TMR implant in the heart wall.

U.S. Pat. No. 6,168,801 describes a cylindrical silicone drug delivery device containing at least two compounds with drug dissolved in them, each compound having different solubility for the drug. The combination of the two different variants of the same drug with different solubility parameters provides the material with control over the rate of drug release.

U.S. Pat. No. 6,053,924 describes a medical device for performing Trans Myocardial Revascularization (TMR) in a human heart. The device consists of a myocardial implant and a directable intracardiac catherter for delivery into a heart wall of an implant. The myocardial implant is used to stimulate angiogenesis in the treated heart wall.

Well-known drug delivery devices include mechanical or electromechanical infusion pumps such as those described in, for example, U.S. Pat. Nos. 4,692,147; 4,360,019; 4,487,603; 4,360,019; 4,725,852, and the like. Osmotically-driven pumps (such as the DUROST™ osmotic pump) are described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; 5,985,305; 5,728,396 and WO 97/27840.

Another well-known drug delivery device is the “depot” which is an injectable biodegradable sustained release device that is generally non-containerized and that may act as a reservoir for a drug, and from which a drug is released. Depots include polymeric and non-polymeric materials, and may be solid, liquid or semi-solid in form. For example, a depot as used in the present invention may be a high viscosity liquid, such as a non-polymeric non-water-soluble liquid carrier material, e.g., Sucrose Acetate Isobutyrate (SAIB) or another compound described in U.S. Pat. Nos. 5,747,058 and 5,968,542, both expressly incorporated by reference herein. For reference, please refer generally to “Encyclopedia of Controlled Drug Delivery” 1999, published by John Wiley & Sons Inc, edited by Edith Mathiowitz.

There has been extensive research in the area of biodegradable controlled release systems for bioactive compounds. Biodegradable matrices for drug delivery are useful because they obviate the need to remove the drug-depleted device. The most common matrix materials for drug delivery are polymers. The field of biodegradable polymers has developed rapidly since the synthesis and biodegradability of polylactic acid was reported by Kulkarni et al., in 1966 (“Polylactic acid for surgical implants,” Arch. Surg., 93:839). Examples of other polymers which have been reported as useful as a matrix material for delivery devices include polyanhydrides, polyesters such as polyglycolides and polylactide-co-glycolides, polyamino acids such as polylysine, polymers and copolymers of polyethylene oxide, acrylic terminated polyethylene oxide, polyamides, polyurethanes, polyorthoesters, polyacrylonitriles, and polyphosphazenes. See, for example, U.S. Pat. Nos. 4,891,225 and 4,906,474 to Langer (polyanhydrides), U.S. Pat. No. 4,767,628 to Hutchinson (polylactide, polylactide-co-glycolide acid), and U.S. Pat. No. 4,530,840 to Tice, et al. (polylactide, polyglycolide, and copolymers).

Degradable materials of biological origin are well known, for example, crosslinked gelatin. Hyaluronic acid has been crosslinked and used as a degradable swelling polymer for biomedical applications (U.S. Pat. No. 4,957,744 to Della Valle et al.; (1991) “Surface modification of polymeric biomaterials for reduced thrombogenicity,” Polym. Mater. Sci. Eng., 62:731-735).

Biodegradable hydrogels have also been developed for use in controlled drug delivery as carriers of biologically active materials such as hormones, enzymes, antibiotics, antineoplastic agents, and cell suspensions. Temporary preservation of functional properties of a carried species, as well as the controlled release of the species into local tissues or systemic circulation, have been achieved. See for example, U.S. Pat. No. 5,149,543 to Cohen. Proper choice of hydrogel macromers can produce membranes with a range of permeability, pore sizes and degradation rates suitable for a variety of applications in surgery, medical diagnosis and treatment.

Many dispersion systems are currently in use as, or being explored for use as carriers of substances, particularly biologically active compounds. Dispersion systems used for pharmaceutical and cosmetic formulations can be categorized as either suspensions or emulsions. Suspensions are defined as solid particles ranging in size from a few nanometers up to hundreds of microns, dispersed in a liquid medium using suspending agents. Solid particles include microspheres, microcapsules, and nanospheres. Emulsions are defined as dispersions of one liquid in another, stabilized by an interfacial film of emulsifiers such as surfactants and lipids. Emulsion formulations include water in oil and oil in water emulsions, multiple emulsions, microemulsions, microdroplets, and liposomes. Micro droplets are unilamellar phospholipid vesicles that consist of a spherical lipid layer with an oil phase inside, as defined in U.S. Pat. Nos. 4,622,219 and 4,725,442 issued to Haynes. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

U.S. Pat. No. 4,938,763 to Dunn, et al., discloses a method for forming an implant in situ by dissolving a non-reactive, water insoluble thermoplastic polymer in a biocompatible, water soluble solvent to form a liquid, placing the liquid within the body, and allowing the solvent to dissipate to produce a solid implant. The polymer solution can be placed in the body via syringe. The implant can assume the shape of its surrounding cavity. In an alternative embodiment, the implant is formed from reactive, liquid oligomeric polymers which contain no solvent and which cure in place to form solids, usually with the addition of a curing catalyst.

Various mechanical means have been used to achieve local drug delivery to the heart. In U.S. Pat. No. 5,551,427, issued to Altman, implantable substrates for local drug delivery at a depth within the heart are described. The patent shows an implantable helical injection needle, which can be screwed into the heart wall and connected to an implanted drug reservoir outside the heart. This system allows injection of drugs directly into the wall of the heart acutely by injection from the proximal end, or on an ongoing basis by a proximally located implantable subcutaneous port reservoir, or pumping mechanism. The patent also describes implantable structures coated with coating, which releases bioactive agents into the myocardium. This drug delivery may be performed by a number of techniques, among them infusion through a fluid pathway, and delivery from controlled release matrices at a depth within the heart. Controlled release matrices are drug polymer composites in which a pharmacological agent is dispersed throughout a pharmacologically inert polymer substrate. Sustained drug release takes place via particle dissolution and slowed diffusion through the pores of the base polymer. Pending U.S. applications Ser. No. 08/881,850 by Altman and Altman, and Ser. No. 09/057,060 by Altman describes some additional techniques for delivering pharmacological agents locally to the heart.

Local drug delivery has been used in cardiac pacing leads. Devices implanted into the heart have been treated with anti-inflammatory drugs to limit the inflammation of the heart caused by the wound incurred while implanting the device itself. For example, pacing leads have incorporated steroid drug delivery to limit tissue response to the implanted lead, and to maintain the viability of the cells in the region immediately surrounding the implanted device. U.S. Pat. No. 5,002,067 issued to Berthelson describes a helical fixation device for a cardiac pacing lead with a groove to provide a path to introduce anti-inflammatory drug to a depth within the tissue. U.S. Pat. No. 5,324,325 issued to Moaddeb describes a myocardial steroid releasing lead whose tip of the rigid helix has an axial bore which is filled with a therapeutic medication such as a steroid or steroid based drug U.S. Pat. Nos. 5,447,533 and 5,531,780 issued to Vachon describe pacing leads having a stylet introduced anti inflammatory drug delivery dart and needle, which is advanceable from the distal tip of the electrode.

U.S. Pat. No. 6,102,887 describes drug delivery catheters that provide a distensible penetrating element such as a helical needle or straight needle within the distal tip of the catheter. The penetrating element is coupled to a reservoir or supply line within the catheter so that drugs and other therapeutic agents can be injected through the penetrating element into the body tissue, which the element penetrates. In use, the drug delivery catheter is navigated through the body to the organ or tissue to be treated, the penetrating element is advanced from the distal end of the catheter, and a therapeutic agent is delivered through the penetrating elements into the organ of tissue. For example, the device may be navigated through the vasculature of a patient into the patient's heart, where the penetrating element is advanced to cause it to penetrate the endocardium, and an anti-arrhythmic drug or pro-rhythmic drug can be injected deep into the myocardium through the penetrating element.

Other Coronary Diseases, Need for Invention, References

Coronary artery disease is just one of many cardiac disease states that has the potential to be treated by delivery of a drug to the heart, over a protracted period, from an implanted device. Other drugs that lend themselves to such treatment include a calcium channel blocker, an antihypertensive agent, an anti-coagulant, an antiarrhythmic agent, an agent to treat congestive heart failure, or a thrombolytic agent (discussed in more detail below).

Arrhythmia and Heart Failure

Cardiac arrhythmias are disorders involving the electrical impulse generating system of the heart. The disorders include premature contractions (extrasystoles) originating in abnormal foci in atria or ventricles, paroxysmal supraventricular tachycardia, atrial flutter, atrial fibrillation, ventricular fibrillation and ventricular tachycardia (Goodman et al, eds., The Pharmacological Basis of Therapeutics, Sixth Edition, New York, MacMillan Publishing Co., pages 761-767 (1980)). More particularly, cardiac arrhythmia is a disorder of rate, rhythm or conduction of electrical impulses within the heart. It is often associated with coronary artery diseases, e.g., myocardial infarction and atherosclerotic heart disease. Arrhythmia can eventually cause a decrease of mechanical efficiency of the heart, reducing cardiac output. As a result, arrhythmia can have life-threatening effects that require immediate intervention.

Perioperative arrhythmias are common. In 2.5% they result in a severe adverse outcome. Various well-known drugs are commonly used to treat arrhythmia (Conway DS et al., Curr Opin Investig Drugs Jan. 2, 2001; (1):87-92). Ventricular arrhythmia is considered as a premonitory sign and risk marker of sudden death. Ventricular tachycardia (VT) is most often associated with structural heart disease: ischemic heart disease and previous myocardial infarction, cardiomyopathy (dilated and hypertrophic), arrhythmogenic right ventricular dysplasia, valvular heart disease (mitral valve prolapse), heart failure, condition after surgical correction of a congenital heart disease. Prognostic significance of VT mostly depends on the type and degree of structural heart disease and on global cardiac function. In patients with asymptomatic non-sustained VT and low risk for sudden death no treatment is needed or antiarrhythmics are administered. Conversely, in high risk patients implantation of automatic cardioverter-defibrillator is indicated. In the treatment of acute attack of VT the following can be used: electroconversion, cardiac pacing (overdrive), lidocaine, amiodarone, beta-blockers, and occasionally magnesium or verapamil. In the prevention of recurrent arrhythmia and sudden death we can use: amiodarone, sotalol, mexiletin, phenytoin, beta-blockers, radiofrequency ablation, implantable cardioverter-defibrillator, and in specific patients verapamil, pacemaker or left ganglion stellatum denervation.

Implantable anti-arrhythmia devices have been developed that employ sophisticated arrhythmia detection and classification methods to accurately determine whether delivery of therapy is appropriate. Particularly in the context of devices such as cardioverters and defibrillators which have the potential to induce arrhythmias if not appropriately synchronized to the patient's heart rhythm, these detection methods tend to be conservative, in order to avoid delivery of unnecessary therapy. In such cases, it may sometimes take the implanted device longer than the patient to determine that delivery of a therapy is needed. Patient activators as discussed above which trigger therapy on request address this problem, but do not provide for the possibility of patient error.

Heart failure is characterized by the inability of the myocardium to shorten sufficiently or to eject an adequate stroke volume to maintain normal perfusion of both the cardiac and the extracardiac organs. The depression of myocardial contractility represents one of the major mechanisms that contributes to low output in heart failure. Beta-receptor-blocking agents (“beta blockers”) have been used in numerous studies for treating the failing heart, especially in dilated cardiomyopathy and ischemic heart disease. In this regard, specific therapeutic aims of the use of beta-receptor-blocking agents in chronic heart failure have been described. e.g., reduction of an increased heart rate in tachycardia, blood pressure reduction in hypertensive heart failure, improvement of supraventricular and ventricular arrhythmias, depression of an increased sympathetic tone (e.g., in hyperthyroidism, phenochromocytoma), increase in the amount of downregulated beta-receptors, and anti-ischemic effects in coronary artery disease. For chronic heart failure, therefore, some special indications may be established and may be individually used; for acute heart failure, only very rare indications are present (e.g., hypertensive crisis, life-threatening cardiac arrhythmias).

Atrial Fibrillation After Cardiac Surgery

Atrial fibrillation occurs in 10% to 65% of patients after cardiac surgery, usually on the second or third postoperative day. Postoperative atrial fibrillation is associated with increased morbidity and mortality and longer, more expensive hospital stays. Prophylactic use of beta-adrenergic blockers reduces the incidence of postoperative atrial fibrillation and should be administered before and after cardiac surgery to all patients without contraindication. Prophylactic amiodarone and atrial overdrive pacing may be considered for patients at high risk for postoperative atrial fibrillation (for example, patients with previous atrial fibrillation or mitral valve surgery). For patients who develop atrial fibrillation after cardiac surgery, a strategy of rhythm management or rate management may be selected. For patients who are hemodynamically unstable or highly symptomatic or who have a contraindication to anticoagulation, rhythm management with electrical cardioversion, amiodarone, or both is preferred. Treatment of the remaining patients is generally focused on rate control because most will spontaneously revert to sinus rhythm within 6 weeks after discharge. All patients with atrial fibrillation persisting for more than 24 to 48 hours and without contraindication are recommended to receive anticoagulation. Thus, Atrial fibrillation frequently complicates cardiac surgery and causes very high additional expense in post-operative hospitalization. However, many cases could be prevented with appropriate prophylactic therapy. A strategy of rhythm management for symptomatic patients and rate management for all other patients usually results in reversion to sinus rhythm within 6 weeks of discharge. See Maisel W H, et al., Ann Intern Med Dec. 18, 2001;135(12):1061-73. If an anti-arrhythmic agent could be directly administered or applied to the heart, it could prevent or diminish post-operative atrial fibrillation and therefore improve treatment, reduce hospitalization time, and reduce cost.

Anti-Arrhythmic Drugs

Anti-arrhythmic drugs are commonly divided into four classes according to their electro-physiological mode of action. See Edvardsson, Current Therapeutic Research, Vol. 28, No. 1 Supplement, pages 113S-118S (July 1980); and Keefe et al, Drugs, Vol. 22, pages 363-400 (1981) for background information of classification first proposed by Vaughn-Williams; see Classification of Anti-Arrhythmic Drugs in Symposium of Cardiac Arrhythmias, pages 449-472, Sandoe et al, (eds.) A. B. Astra, Soederlalje, Sweden (1970).

The classification of anti-arrhythmic drugs is as follows:

I. Local anesthetic effect

II. Beta-receptor blockade

III. Prolongation of action potential duration

IV. Calcium antagonism.

Class I agents usually have little or no effect on action potential duration and exert local anesthetic activity directly at cardiac cell membrane. Class II agents show little or no effect on the action potential and exert their effects through competitive inhibition of beta-adrenergic receptor sites, thereby reducing sympathetic excitation of the heart. Class III agents are characterized by their ability to lengthen the action potential duration, thereby preventing or ameliorating arrhythmias. Class IV agents are those which have an anti-arrhythmic effect due to their actions as calcium antagonists.

Class I

Sodium Channel Depressors

These agents are efficacious in repressing a sodium current. However, these agents have no or only minute effects on the retention time of the normal action potential and decrease the maximum rising velocity (V.sub.max) of the sodium current. They exert anti-arrhythmic activity but at the same time strongly repress cardiac functions. Careful consideration is required in administering to patients with cardiac failure or hypotension.

Class II

Beta-Blocking Agents

The agents in this class, represented by propranolol, are efficacious in the beta-blocking action and are useful in treating patients with arrhythmia in which the sympathetic nerve is involved. However, care must be taken in their use since these agents have side effects caused by the beta-blocking action, such as depression of cardiac functions, induction of bronchial asthmatic attack and hypoglycemic seizures.

Class III

Pharmaceutical Agents for Prolonging the Retention Time of the Action Current

These agents are efficacious in remarkably prolonging the retention time of the action current of the cardiac muscle and in prolonging an effective refractory period. Re-entry arrhythmia is considered to be suppressed by the action of the pharmaceutical agents of Class III. The medica-ments of this Class III include amiodarone and bretylium. However, all the agents have severe side effects; therefore, careful consideration is required for use.

Class IV

Calcium Antagonists

These agents control a calcium channel and suppress arrhythmia due to automatic asthenia of sinoatrial nodes and to ventricular tachycardia in which atrial nodes are contained in the re-entry cycle.

Although various anti-arrhythmic agents within the above classes are now available on the market, those having both satisfactory effects and high safety have not been obtained. For example, anti-arrhythmic agents of Class I which cause a selective inhibition of the maximum velocity of the upstroke of the action potential (V.sub.max) are inadequate for preventing ventricular fibrillation. In addition, they have problems regarding safety, namely, they cause a depression of the myocardial contractility and have a tendency to induce arrhythmias due to an inhibition of the impulse conduction. Beta-adrenoceptor blockers and calcium antagonists which belong to Classes II and IV, respectively, have the defect that their effects are either limited to a certain type of arrhythmia or are contraindicated because of their cardiac depressant properties in certain patients with cardiovascular disease. Their safety, however, is higher than that of the anti-arrhythmic agents of Class I.

Anti-arrhythmic agents of Class III are drugs which cause a selective prolongation of the duration of the action potential without a significant depression of the V.sub.max. Drugs in this class are limited. Examples such as sotalol and amiodarone have been shown to possess Class III properties. Sotalol also possesses Class II effects which may cause cardiac depression and are contraindicated in certain susceptible patients. Also, amiodarone is severely limited by side effects. Drugs of this class are expected to be effective in preventing ventricular fibrillations. Pure Class III agents, by definition, are not considered to cause myocardial depression or an induction of arrhythmias due to the inhibition of the action potential conduction as seen with Class I anti-arrhythmic agents.

A number of anti-arrhythmic agents have been reported in the literature, such as those disclosed in EP 397,121; EP 300,908; EP 307,121; U.S. Pat. Nos. 4,629,739; 4,544,654; 4,788,196; EP application 88 302 597.5; EP application 88 302 598.3; EP application 88 302 270.9; EP application 88 302 600.7; EP application 88 302 599.1; EP application 88 300 962.3; EP application 235,752; DE 36 33 977; U.S. Pat. Nos. 4,804,662; 4,797,401; 4,806,555; and 4,806,536.

None of the previous approached provide a biodegradable, non-polymer depot that can be implanted into cardiac tissue to effect sustained delivery of a drug such as an antiarrhythmic factor or an angiogenic factor, such as VEGF or FGF.

For background literature generally, see: Lazarous et al. (1997) Cardiovascular Research 36:78-85; and Landau et al. (1995) Am. Heart. J. 129:924-931; Laham et al. (2000) J. Pharm. Exp. Ther. 292:795-802. U.S. Pat. Nos. 5,387,419; 5,931,810; 5,827,216; 5,900,433; 5,681,278; 6,251,418; 5,634,895; 5,387,419 and 5,797,870; and PCT Publication No. WO 97/16170. U.S. Pat. Nos. 6,187,330; 6,238,408; and 6,152,141.

### SUMMARY OF THE INVENTION

Objects and Overview of the Invention—Myocardial Implants

The following invention information was first presented in U.S. patent application Ser. No. 60/347,326 filed Jan. 9, 2002. Herein incorporated by reference.

The present invention encompasses compositions and methods providing sustained-release of a drug to the heart or coronary vasculature using an implanted dosage form that may be implanted in the cardiac or vascular tissue, or that may be implanted at another site, but designed to supply a drug to the heart or vasculature via a catheter, or that may be sprayed directly onto the heart. The drug delivered may be any type of drug, such as angiogenic agents, calcium channel blockers, antihypertensive agents, beta-blockers, anti-arrhythmic agents, steroids, antibodies or anti-proliferatines.

In particular, the invention is directed to a pump or a biodegradable implant or to a depot, such as a depot comprising a non-polymeric, high viscosity material, e.g., Sucrose Acetate Isobutyrate (SAIB) or another compound described in U.S. Pat. Nos. 5,747,058 and 5,968,542. Such non-polymeric high viscosity material acts as a carrier material and is generally considered liquid in consistency. In a specific embodiment the depot may contain an angiogenic factor such as VEGF or fibroblast growth factor (FGF) or an antiarrhythmic agent.

Pumps are generally implanted subcutaneously, for example in the chest area, under the arm, and employ a catheter threaded through the chest wall and implanted in the myocardium. Depots generally are injected directly into the myocardial tissue, but may also be sprayed onto the heart tissue directly. This is of particular interest when delivering antiarrhythmic agents.

The present invention provides methods useful for treating any manner of cardiac disease, such as arrhythmia, or for increasing cardiac function by increasing vascularization by encouraging angiogenesis. The methods generally involve using a sustained-release dosage form to deliver a drug into the myocardial or vascular tissue at a low volume and/or low dosage rate.

The methods are particularly useful when delivery of a drug to the cardiac tissue is desired for an extended period of time to increase its effectiveness or to reduce the risk and/or severity of adverse side effects, or to reduce the amount (and therefore cost) of drug delivered.

In various aspects, the drug may be delivered at a low dose rate, e.g., up to about 0.01 microgram/hr, 0.10 microgram/hr, 0.25 microgram/hr, 1 microgram/hr, or 5, 10, 25, 50, 75, 100, 150, or generally up to about 200 microgram/hr. Specific ranges of amount of drug delivered will vary depending upon, for example, the potency. In one exemplary embodiment, a drug formulation is delivered at a low volume rate e.g., a volume rate of from about 0.01 microliters/day to about 2 ml/day. Delivery of a formulation can be substantially continuous or pulsate, and can be for a pre-selected administration period ranging from several hours to years.

The sustained release drug delivery devices can be any device, e.g., osmotic pumps (used with or without a catheter), biodegradable implants, electrodiffusion systems, electroosmosis systems, vapor pressure pumps, electrolytic pumps, effervescent pumps, piezoelectric pumps, electrochemical pumps, erosion-based systems, depots, microspheres, or electromechanical systems.

Cardiac conditions which are amenable to treatment according to the invention include any pathological conditions, especially a condition of the heart that is amenable to treatment by increasing the number of functional coronary blood vessels, e.g., an ischemic heart disease; cardiac arrhythmia; a cardio-myopathy; coronary angioplasty restenosis; myocardial infarction; atherosclerosis of a coronary artery; thrombosis; a cardiac condition related to hypertension; cardiac tamponade; and pericardial effusion.

The present invention takes advantage of sustained-release delivery technology in the form of miniature pumps and in the form of depots and implants. Where a pump is used, it will generally be implanted subcutaneously, for example in the chest wall or under the arm, and will employ a catheter to deliver drug, where the distal end of which is implanted into cardiac tissue and held in place by sutures. An osmotic pump will likely not be implanted directly into the myocardial tissue because of eh relative scarcity of interstitial water required to activate the osmotic pump. Additionally, the invention employs a non-polymeric depot that can be injected into a tissue to effect sustained release of a specific drug locally, producing highly effective local concentrations of a drug, but without the undesirable sire-effects of systemic drug delivery. The non-polymeric depot, having released the drug for the desired period, is slowly degraded by the body, overcoming the need to remove the drug delivery device.

Generally, embodiments of the invention include a method for improving cardiac function in a subject, the method comprising: implanting in said subject a sustained release dosage form, said sustained release dosage form comprising a drug delivery device and a cardiac drug, and administering said cardiac drug from said dosage form into said subject, for a period of at least 24 hours, in an dose sufficient to cause a measurable improvement in cardiac function. Also included are methods wherein the dosage form is placed in the pericardial sac, or implanted within the myocardial tissue, or sprayed directly onto the heart. The drug delivery device can be a pump, or bioerodable implant, or depot. Generally, the cardiac drug is selected from the group consisting of: an angiogenic factor, growth factor, calcium channel blocker, antihypertensive agent, inotropic agent, antiatherogenic agent, anti-coagulant, beta-blocker, anti-arrhythmic agent, anti-inflammatory agent, sympathomimetic agent, phosphodiesterase inhibitor, diuretic, vasodilator, thrombolytic agent, cardiac glycoside, antibiotic, antiviral agent, antifungal agent, antineoplastic agent, and steroid.

Advantages of the Invention

An advantage of the present invention is that relatively small quantities of a drug can be administered over an extended period of time to the heart tissues. The methods of the present invention thus avoid the pitfalls associated with systemic delivery of a drug.

A further advantage of the present invention is that it avoids problems associated with bolus injection of a drug, such as delivery of an amount of drug to the cardiac tissue which is too high and which therefore may have deleterious effects on the cardiac tissue.

Another advantage is that it provides long-term delivery of a drug to the pericardium or myocardial tissue, with even delivery rate, approximating to zero-order kinetics over a substantial period of delivery.

Another important advantage is that extended delivery of a drug to the cardiac tissue can be achieved without the need for repeated invasive surgery, thereby reducing trauma to the patient.

Another advantage is that the depot eventually degrades, obviating the need for removal.

These and other objects, advantages, and features of the invention will become apparent to those persons skilled in the art upon reading the details of the invention as more fully described below.

Objects and Overview of the Invention—Pericardial Delivery

The following invention information was first presented in U.S. patent applications Ser. Nos. 60/278,518 and 60/311,309 filed Mar. 23, 2001 and Aug. 09, 2001 respectively, both herein incorporated by reference.

The present invention also provides compositions and methods that involve introducing a cardiac drug into the pericardial space at a low volume and/or low dosage rate. The methods are useful in treating a variety of cardiac disease conditions, e.g., ischemia. The methods are particularly useful for drug delivery over an extended period of time for example, for delivery of drug at a low volume rate to reduce the risk, incidence, and/or severity of adverse side effects. Introduction of the cardiac drug into the pericardial space can be via transpericardial or intrapericardial routes. The condition being treated may be an ischemic or arrhythmic condition, and the cardiac drug being delivered can be an angiogenic factor, e.g. fibroblast growth factor (FGF) or an anti-arrhythmic, e.g., a beta blocker. In many embodiments, the cardiac drug may be an angiogenic factor or anti-arrhythmic factor. Angiogenic factors increase coronary blood flow as a result of an increase in the number of functional collateral blood vessels. Anti-arrhythmic factors correct abnormal rhythms frequently associated with abnormal impulse generation.

In various aspects thereof, the cardiac drug of the drug formulation administered is delivered at a low dose rate, e.g., from about 0.01 μg/hr or 0.1 μg/hr, 0.25 μg/hr, 1 μg/hr, generally up to about 10, 50, 100, 150, or 200 μg/hr.

In one exemplary embodiment, a drug formulation comprising a cardiac drug is delivered at a low volume rate e.g., a volume rate of from about 0.01 μl/day to about 2 ml/day.

In another exemplary embodiment, delivery of a formulation comprising a cardiac drug is substantially continuous, and can be for a pre-selected administration period ranging from several hours to years.

Cardiac conditions which are amenable to treatment according to the invention include any abnormal or pathological condition of the heart that is amenable to treatment by increasing the number of functional coronary blood vessels, e.g., an ischemic heart disease; cardiac arrhythmia; a cardiomyopathy; coronary angioplasty restenosis; myocardial infarction; atherosclerosis of a coronary artery; thrombosis; a cardiac condition related to hypertension; cardiac tamponade; and pericardial effusion.

The present invention takes advantage of the phenomenon that drug delivered to the pericardial fluid primarily enters the systemic circulation by crossing the epicardium and entering the myocardial tissue, rather than by crossing the pericardium.

A primary object of the invention is to provide a method for convenient, long-term management of a condition, particularly a cardiac condition.

An advantage of the methods of the present invention is that relatively small quantities of a cardiac drug can be administered over an extended period of time to the pericardial space. The methods of the present invention thus avoid the pitfalls associated with systemic delivery of a cardiac drug, namely that high systemic doses are often required to achieve an effective dose in the cardiac tissue (which effective dose is much lower than the systemic dose delivered), and such high systemic doses may have deleterious effects on non-cardiac tissues. A further advantage of the methods of the present invention is that relatively low doses of a cardiac drug can be delivered over a period of time to the cardiac tissue, thereby avoiding problems associated with bolus injection of a cardiac drug, such as delivery of an amount of drug to the cardiac tissue which is too high and which therefore may have deleterious effects on the cardiac tissue.

The methods of the present invention are further advantageous in that long-term delivery of a cardiac drug to the pericardial space can be achieved. This aspect is particularly useful in cases in which the beneficial effects of a cardiac drug are achieved only when a cardiac drug is administered over an extended period of time.

Another important advantage of the methods of the present invention is that extended delivery of a cardiac drug to the cardiac tissue can be achieved without the need for repeated invasive surgery, thereby reducing trauma to the patient.

These and other objects, advantages, and features of the invention will become apparent to those persons skilled in the art upon reading the details of the invention as more fully described below.

Notice Regarding Limitations

Before the present invention is further described, it is to be understood that this invention is not limited to particular embodiments described. It is also to be understood that the terminology used herein is for the purpose of describing particular embodiments only, and is not intended to be limiting, since the scope of the present invention will be limited only by the appended claims. Where a range of values or a number is provided, it is understood that the range or number includes half values either side of a stated number. Unless defined otherwise, all technical and scientific terms used herein have the same meaning as commonly understood by one of ordinary skill in the art. All publications mentioned herein are incorporated herein by reference to disclose and describe the methods and/or materials in connection with which the publications are cited. Please note that the singular forms “a”, “and”, and “the” include plural referents unless the context clearly dictates otherwise. Thus, for example, reference to “an angiogenic factor” includes a plurality of such factors.

### BRIEF DESCRIPTIONS OF THE DRAWINGS

FIG. 1 is a bar graph depicting indexed heart weights, expressed as mg heart weight per gram total body weight, of SHR and RSA rats treated with FGF-2 via iv infusion, ipc bolus injection, or ipc infusion. WKY rats served as normal controls for heart weight.

FIG. 2 is a bar graph depicting cardiac capillary densities in rats treated with FGF-2/heparin.

FIG. 3 is a bar graph depicting coronary conductance of SHR rat hearts with FGF-2 or RSA via iv infusion, ipc bolus injection, or ipc infusion. WKY rats served as normal controls.

FIG. 4(A-H) is a collection of graphs depicting concentration-time profiles of fluorescent macromolecules in rat pericardial fluid after intrapericardial bolus injection (FIGS. 4A-D) or in plasma after intra-arterial bolus injection (FIGS. 4E-H).

FIG. 5(A-D) is a collection of graphs depicting the ratios of fluorescence measured in pericardial fluid and plasma after intra-pericardial (closed symbols) or intra-arterial bolus injections of fluorescent macromolecules.

FIG. 6 a graph depicting the concentration of Texas red-labeled rat albumin, administered by infusion into the pericardial space, at various times after the start of infusion.

FIG. 7 is a graph depicting the ratio of the concentration of albumin in the pericardial fluid to the concentration of albumin in plasma over the 7-day infusion period.

FIG. 8 is a graph depicting the concentration of Texas red-labeled bFGF, administered by infusion into the pericardial space, at various times after the start of infusion.

FIG. 9 is a graph depicting the ratio of the concentration of bFGF in the pericardial fluid to the concentration of bFGF in plasma over the 7-day infusion period.

FIG. 10 is a graph depicting the concentration of cortisol, administered by infusion into the pericardial space, at various times after the start of infusion.

FIG. 11 is a graph depicting the ratio of the concentration of cortisol in the pericardial fluid to the concentration of albumin in cortisol over the 7-day infusion period.

### DEFINITIONS

The term “cardiac condition” as used herein, refers to any abnormal or pathological condition of the heart that is amenable to treatment with a drug, including, but not limited to, an arrhythmia or an ischemic heart disease (due to, e.g., cardiac hypertrophy, atherosclerosis, a cardiomyopathy, hyperthyroidism, and the like); cardiac arrhythmia; a cardiomyopathy; coronary angioplasty restenosis; myocardial infarction; atherosclerosis of a coronary artery; thrombosis; a cardiac condition related to hypertension; cardiac tamponade; and pericardial effusion.

The phrase “increasing cardiac function” includes increasing, to any measurable degree myocardial and coronary blood flow, increase in left ventricular function, increase in local functional (wall motion) analysis, decrease in ischemic area, increase in myocardial perfusion score, favorable change in the unipolar and bipolar endocardial potentials reflective of myocardial viability, and electrocardiographic normalization; the term also includes reduction in arrhythmia.

The term “cardiac vasculature” refers to the arteries and veins immediately attached to the heart, including, but not limited to the aorta, brachiocephalic artery, left common carotid artery, left subclavian artery, superior and inferior vena cava, right and left pulmonary artery, right and left pulmonary veins, pulmonary trunk, left and right coronary artery, left and right coronary vein, cardiac arteries including grand cardiac vein, circumflex artery, coronary sinus, posterior and anterior descending coronary artery, right and left anterior descending artery, and any and all veins and arteries that transport blood to and from the myocardial tissue.

The term “sustained release” means release (of a drug) over an extended period of time, as contrasted with an all-at-once “bolus” release. Sustained release, for example, may be for a period of at least 12 hours, at least 24 hours, at least two weeks, at least a month, at least three months, or longer.

The term “drug delivery device” refers to any means for containing and releasing a drug wherein the drug is released into a subject. Drug delivery devices are split into five major groups: inhaled, oral, transdermal, parenteral and suppository. Inhaled devices include gaseous, misting, emulsifying and nebulizing bronchial (including nasal) inhalers; oral includes mostly pills; whereas transdermal includes mostly patches. Parenteral includes two sub-groups: injectable and non-injectable devices. Non-injectable devices are generally referred to as “implants” or “non-injectable implants” and include e.g., pumps and solid biodegradable polymers. Injectable devices are split into bolus injections, that are injected and dissipate, releasing a drug all at once, and depots, that remain discrete at the site of injection, releasing drug over time. Depots include e.g., oils, gels, liquid polymers and non-polymers, and microspheres. Many drug delivery devices are described in Encyclopedia of Controlled Drug Delivery (1999), Edith Mathiowitz (Ed.), John Wiley & Sons, Inc.

The term “dosage form” refers to a drug plus a drug delivery device.

The term “microspheres” (also known as “microparticles” or nanospheres” or “nanoparticles”) refers to small particles, typically prepared from a polymeric material and typically no greater in size than about 10 micrometer in diameter. (“Encyclopedia of Controlled Drug Delivery” 1999, published by John Wiley & Sons Inc, edited by Edith Mathiowitz.) For example, U.S. Pat. No. 4,994,281 discloses polylactic acid microspheres, prepared by the in-water drying method, containing a physiologically active substance and having an average particle size of about 0.1 to 10 micrometers.

The term “formulation” means any drug together with a pharmaceutically acceptable excipient or carrier such as a solvent such as water, phosphate buffered saline or other acceptable substance. A formulation may include one or more cardiac drugs, and also encompass one or more carrier materials such as SAIB or other carrier materials such as described in U.S. Pat. Nos. 5,747,058 and 5,968,542.

The term “drug” as used herein, refers to any substance meant to alter animal physiology.

The term “cardiac drug” refers to any drug meant to alter the physiology of a mammalian heart, and includes, but is not limited to: angiogenic factors, growth factors, calcium channel blockers, antihypertensive agents, inotropic agents, antiatherogenic agents, anti-coagulants, beta-blockers, anti-arrhythmic agents, anti-inflammatory agents, sympathomimetic agents, phosphodiesterase inhibitors, diuretics, vasodilators, thrombolytic agents, cardiac glycosides, antibiotics, antiviral agents, antifungal agents, agents that inhibit protozoans, antineoplastic agents, and steroids.

The term “arrhythmia” means any pathology of rate, rhythm or conduction of electrical impulses within the heart.

The term “anti-arrhythmia agent” or “anti-arrhythmic” refers to any drug used to treat a disorder of rate, rhythm or conduction of electrical impulses within the heart (see Background).

The term “angiogenic agent” (or “angiogenic factor”) means any compound that promotes growth of new blood vessels. Angiogenic factors include, but are not limited to, a fibroblast growth factor, e.g., basic fibroblast growth factor (bFGF), and acidic fibroblast growth factor, e.g., FGF-1, FGF-2, FGF-3, FGF-4, recombinant human FGF (U.S. Pat. No. 5,604,293); a vascular endothelial cell growth factor (VEGF), including, but not limited to, VEGF-1, VEGF-2, VEGF-D (U.S. Pat. No. 6,235,713); transforming growth factor-alpha; transforming growth factor-beta; platelet derived growth factor; an endothelial mitogenic growth factor; platelet activating factor; tumor necrosis factor-alpha; angiogenin; a prostaglandin, including, but not limited to PGE₁, PGE₂; placental growth factor; GCSF (granulocyte colony stimulating factor); HGF (hepatocyte growth factor); IL-8; vascular permeability factor; epidermal growth factor; substance P; bradykinin; angiogenin; angiotensin II; proliferin; insulin like growth factor-1; nicotinamide; a stimulator of nitric oxide synthase; estrogen, including, but not limited to, estradiol (E2), estriol (E3), and 17-beta estradiol; and the like. Angiogenic factors further include functional analogs and derivatives of any of the aforementioned angiogenic factors. Derivatives include polypeptide angiogenic factors having an amino acid sequence that differs from the native or wild-type amino acid sequence, including conservative amino acid differences (e.g., serine/threonine, asparagine/glutamine, alanine/valine, leucine/isoleucine, phenylalanine/tryptophan, lysine/arginine, aspartic acid/glutamic acid substitutions); truncations; insertions; deletions; and the like, that do not substantially adversely affect, and that may increase, the angiogenic property of the angiogenic factor. Angiogenic factors include factors modified by polyethylene glycol modifications (“PEGylation”); acylation; acetylation; glycosylation; and the like. An angiogenic factor may also be a polynucleotide that encodes the polypeptide angiogenic factor. Such a polynucleotide may be a naked polynucleotide or may be incorporated into a vector, such as a viral vector system such as an adenovirus, adeno-associated virus or lentivirus systems.

“Continuous delivery” as used herein is meant to refer to delivery of a desired amount of substance into the tissue over a period of time, as opposed to bolus delivery.

“Controlled release” as used herein (e.g., in the context of “controlled drug release” and in reference to controlled release drug delivery devices) is meant to encompass release of substance (e.g., a drug) at a selected or otherwise controllable rate, interval, and/or amount.

“Patterned” or “temporal” as used in the context of drug delivery is meant to encompass delivery of drug in a pattern, generally a substantially regular pattern, over a pre-selected period of time.

The term “therapeutically effective amount” is an amount of a therapeutic agent, or a rate of delivery of a therapeutic agent, effective to facilitate a desired therapeutic effect. The precise desired therapeutic effect will vary according to the condition to be treated, the formulation to be administered, and a variety of other factors that are appreciated by those of ordinary skill in the art.

The terms “subject,” “individual,” and “patient,” used interchangeably herein, refer to any subject, generally a mammal (e.g., human, canine, feline, equine, bovine, ursine, icthiine, porcine, ungulate etc.), to which a drug is delivered.

The term “ambient conditions” as used in the present application means normal room temperature and pressure.

The term “physiological conditions” as used in the present application means environmental conditions as usually found within the body of an animal.

### DETAILED DESCRIPTION OF THE INVENTION

The present invention is directed to delivery of a drug to the heart, or to the vessels of the heart by use of a sustained-release drug dosage form implanted in or near the cardiac or vascular tissue or within the pericardial space, or sprayed directly onto the heart surface.

In particular, the invention is directed to an implanted pump (with or without a catheter) or to a depot comprising a non-polymeric, high viscosity liquid carrier material, e.g., Sucrose Acetate Isobutyrate (SAIB) or another compound described in U.S. Pat. Nos. 5,747,058 and 5,968,542.

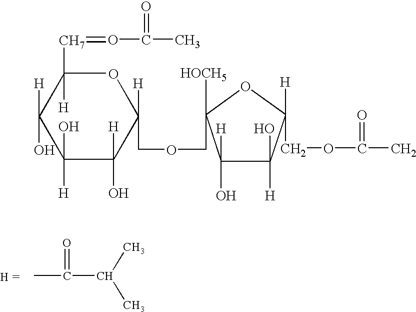

Sucrose Acetate Isobutyrate Chemical Structure

In a specific embodiments the depot may contain an angiogenic factor such as, but not limited to VEGF or fibroblast growth factor (FGF).

Other specific embodiments include a depot containing a calcium channel blocker, an antihypertensive agent, an anti-coagulant, an antiarrhythmic agent, an agent to treat congestive heart failure, or a thrombolytic agent (discussed in more detail below).

Partly, the invention is instigated by the discovery that delivery of an angiogenic factor to the heart interpericardially results in an increase in coronary blood flow, and that infusion provides significantly better results than bolus injection (see EXAMPLES). Increased coronary blood flow results from an increase in the number of functional blood vessels. Intravenous infusion does not achieve this effect. Moreover, bolus administration into the myocardial tissue is not as effective and has deleterious effects in that such administration results in cardiac hypertrophy. This result was unexpected in view of teachings in the art that bolus administration of angiogenic factors into the myocardial tissue achieves increased cardiac function.

The present invention also takes advantage of the discovery that a depot may be formulated to release an angiogenesis factor over a prolonged period with a particularly advantageous drug release profile, and that such a depot may be implanted in the myocardial or vascular tissue where it will effect local delivery of a drug at a desired rate for a desired time.

An example of formulation of a depot of the invention is a depot comprising sucrose acetate isobutyrate (SAIB). A formulation is prepared by mixing SAIB (Eastman Chemical Co.) and benzyl benzoate (Aldrich Chemical Co.) and DL-PLG (or DLPL) in a ratio of 83:12:5 (weight basis) and stirring until a homogeneous mixture is achieved. 10 μg of human, recombinant Fibroblast Growth Factor (FGF) (Sigma Chemical Co.) is then added to 500 μL of the SAIB:benzyl benzoate:DL-PLG formulation and mixed to form an injectable depot. Some examples of additional depot compositions are set out below.

| | SAIB, | Solvent, | Additive, | % Release, | % Release, |
|---|---|---|---|---|---|
| DRUG | % wt | % wt | % wt | 24 h | 168 h |
| VEGF | 65 | DMSO 35% | — | 15 | 70 |
| VEGF | 65 | DMSO 30% | DL-PLG, | 5 | 40 |
| | | | 5% | | |
| FGF | 60 | Benzyl | — | 40 | 85 |
| | | benzoate | | | |
| | | 40% | | | |
| FGF | 60 | Benzyl | DL-PLA, | 20 | 50 |
| | | alcohol 20%/ | 5% | | |
| | | Ethanol 15% | | | |

Other solvents that can be used with SAIB include ethanol, benzoyl benzoate, propylene carbonate, migllyol 801, NMP and DMSO.

In one embodiment, spray freeze-dried rhVEGF powder (10 mg/mL protein, 1.0 mg.mL Trehalose, 0.01% Polysorbate 20) is physically incorporated into a SAIB/solvent solution and homogenized by passing the suspension through a twin hub 18 gauge stainless steel needle.

In other embodiments, directed to gene therapy applications, the implanted dosage form may deliver into a cell a polynucleotide that expresses an angiogenic factor. Such a gene may be engineered, using methods well-known in the art into a suitable mammalian expression vector such as a viral vector such as an adenoviral vector (see U.S. Pat. No. 5,478,745) or an adeno-associated viral vector (see U.S. Pat. Nos. 5,354,687 and 5,474,935) or a lentiviral vector (see U.S. Pat. Nos. 6,207,455; 6,165,782 and 5,994,136). Other gene therapy delivery methods include delivery of polynucleotides or polynucleotides engineered into expression vectors, delivered to a cell as naked polynucleotide, or using liposomes, microspheres or synthetic capsid systems.

Methods for Increasing Cardiac Function by Myocardial Implantation

The present invention provides methods for increasing cardiac function in an individual. The methods generally comprise delivering a drug via a sustained-release dosage form into myocardial tissue.

The drug is generally delivered at a low volume rate of from about 0.01 microliter/day to about 2 ml/day, from about 0.04 microliter/day to about 1 ml/day, from about 0.2 microliter /day to about 0.5 ml/day, or from about 2.0 microliter /day to about 0.25 ml/day.

The desired volume rate of delivery can be adjusted according to a variety of factors, including, for example, the concentration and potency of the drug formulation, as discussed above. Such adjustments are routine to those of ordinary skill in the art.

In general, administration of a drug can be sustained for at least several hours (e.g., 2, 12, 24, 48, 72 hours or more), to at least several days (e.g., 2, 5, 7, 14, 30 days or more), to at least several months (1, 3, 6, 12 months) or years. Typically, delivery can be continued for a period of at least a week, at least 1 month or at least 3 months or more. Delivery of a drug may be in a patterned fashion, or in a substantially continuous, constant rate.

Increase in capillary density is readily determined by those skilled in the art. Capillary density per square millimeter of cardiac tissue in the epicardium can be determined using any known method, including, but not limited to, staining with lectin (e.g., Griffonia simplicifolia).

Increase in coronary blood flow is measured using any known method, including, but not limited to: (1) retrograde Langendorff perfusion (for animals), e.g., in the presence of nitroprusside/adenosine; (2) clearance methods which involve introducing an inert gas (usually nitrous oxide) into the circulation via the lungs and following the progressive saturation of cardiac tissue. The increases in the systemic arterial and coronary sinus concentrations of indicator are measured over the time until arteriovenous difference reaches zero. The reciprocal of this time reflects the blood flow in milliliters per minute per 100 g of tissue; (3) Thermodilution, in which a catheter is passed into the coronary sinus and a continuous infusion of cold saline is made through a lumen near the tip at a constant rate. The temperature of the blood at a site several centimeters back from the tip of the catheter is measured with a thermistor. The method uses the form of the Fick equation dealing with continuous (rather than bolus) infusion of indicator: Q=I/C where Q is the blood flow in ml/min, I the rate of infusion and C the steady level of indicator (temperature difference) resulting from infusion; (4) flow meter techniques, including, e.g., electromagnetic and Doppler flow meters which have been used in surgery, where they are best suited for measurement of the flow in vein grafts, and catheter-tip flow meters which are small enough to enter the large coronary arteries. Laser Doppler probes can potentially measure flow velocity in intramyocardial vessels.

Desired rate of drug delivery depends on several factors, including: (1) the potency of the drug being delivered; (2) the pharmaceutically effective dosage window of the drug, i.e., the dose at which the drug is efficacious without substantial adverse effect; and (3) the pharmacokinetics of the particular drug being delivered, which may be a function of the physical and/or chemical characteristics of the drug.

In particular embodiments of interest, the drug is an angiogenic factor. Thus, the present invention provides methods for increasing cardiac function by delivering an angiogenic factor at low volume rates to the pericardium or myocardial tissue.

In certain embodiments directed to gene therapy applications, the implanted dosage form may deliver into a cell a polynucleotide that expresses an angiogenic factor or anti-arrhythmia agent. Such a gene may be engineered, using methods well-known in the art into a suitable mammalian expression vector such as a viral vector such as an adenoviral vector (see U.S. Pat. No. 5,478,745) or an adeno-associated viral vector (see U.S. Pat. Nos. 5,354,687 and 5,474,935) or a lentiviral vector (see U.S. Pat. Nos. 6,207,455; 6,165,782 and 5,994,136). An example of a polynucleotide encoding an angiogenesis factor is the human VEGF-encoding polynucleotide Accession No. AY047581 (Version AY047581.1 GI:15422108). Another example of a polynucleotide encoding an angiogenesis factor is the human FGF-encoding polynucleotide Accession No. AF411527 (Version AF411527.1 GI: 15705914). In certain applications it may well be desirable to use chromosomal rather than cDNA since the chromosomal version contains introns as well as exons that may be important for proper expression. The desired polynucleotide may be inserted into an appropriate expression vector, i.e., a vector that contains the necessary elements for transcriptional and translational control of the inserted coding sequence in a suitable (mammalian) host. These elements include regulatory sequences, such as enhancers, constitutive and inducible promoters, and 5′ and 3′ untranslated regions in the vector and in polynucleotide sequences encoding the desired protein. Specific initiation signals may also be used to achieve more efficient translation of sequences encoding ABBR. Such signals include the ATG initiation codon and adjacent sequences, e.g. the Kozak sequence. In cases where sequences encoding the desired protein and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. The efficiency of expression may be enhanced by the inclusion of enhancers appropriate for the particular host cell system used. (See, e.g., Scharf, D. et al. (1994) Results Probl. Cell Differ. 20:125-162.).

Methods which are well known to those skilled in the art may be used to construct expression vectors containing sequences encoding the desired protein and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17.

A variety of expression vector/host systems may be utilized to contain and express sequences encoding the desired protein. In mammalian cells, a number of viral-based expression systems may be utilized. For example, in cases where an adenovirus is used as an expression vector, sequences encoding the desired protein may be ligated into an adenovirus transcription/translation complex consisting of the late promoter and tripartite leader sequence. Insertion in a non-essential E1 or E3 region of the viral genome may be used to obtain infective virus which expresses the desired protein in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Alternatively, human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355).

In gene therapy applications, an engineered expression vector is released from a sustained-release dosage form into the tissue in which the dosage form is implanted. The vector transforms the cells of the surrounding local tissue and expresses the desired protein therein. The sustained release dosage form may be, for example, a pump, or a depot, such as an SAIB depot.

Alternatively, polynucleotides may be delivered using liposomes, microspheres or synthetic capsid systems. (See An Introduction To Molecular Medicine And Gene Therapy Thomas F. Kresina, John Wiley & Sons 2000; Li et al, Acta Anaesthesiol Sin December 2000;38 (4):207-15; Kawauchi et al, Gene therapy for attenuating cardiac allograft arteriopathy using ex vivo E2F decoy transfection by HVJ-AVE-liposooume method in mice and nonhuman primates. Circ Res. Nov. 24, 2000; 87 (11):1063-8; and Jayakamur et al, Gene therapy for myocardial protection: transfection of donor hearts with heat shock protein 70 gene protects cardiac function against ischemia-reperfusion injury. Circulation. Nov. 7, 2000;102(19 Suppl 3):III302-6.). For liposome technology see Dalesandro et al, Gene therapy for donor hearts: ex vivo liposome-mediated transfection. J Thorac Cardiovasc Surg. February 1996; 111(2):416-21; and Romero et al, Medicina (B Aires) 2001; 61(2):205-14.). When introduced into a cell, the polynucleotide is expressed to produce an angiogenic protein such as FGF.

Methods of Treating an Individual by Pericardial Delivery

In some embodiments, the subject being treated is catheterized such that a distal end of a catheter, or a distal extension thereof, delivers a pharmaceutically active agent to the pericardial space from the exterior of the heart, either through the pericardium (transpericardial delivery) or directly into the pericardial space (intrapericardial delivery). A drug delivery device, e.g., a controlled release delivery device, is attached to the proximal end of the catheter and effects controlled delivery of the drug to the pericardium and/or into the pericardial fluid.

In one exemplary embodiment, the drug is an angiogenic factor, and the drug delivery device is a pump, e.g., an osmotic pump, which pump is attached to a catheter. A small incision is made in the pericardium, and the catheter is threaded therethrough. A loop, or knot is made in the catheter, and the catheter is threaded through the incision, such that the loop is on the inside of the pericardial sac. The incision is then sewn to leave a hole just large enough for the catheter to fit through, but too small for the loop to slide back out of, thereby securing the catheter in place. The pump is implanted subcutaneously at any convenient location. The pump may be secured by stitching. Drug is supplied from the pump, via the catheter, into the pericardial space, from which is contacts and enters the cardiac tissue.

In another exemplary embodiment, the drug is an angiogenic factor, and the drug delivery device is a depot, e.g. a high viscosity liquid, such as a non-polymeric non-water-soluble liquid carrier material, e.g., sucrose acetate isobutyrate (SAIB) or another compound as described in U.S. Pat. No. 5,747,058. The depot may be formulated using methods well known in the art to achieve the desired physical properties, e.g., of viscosity and rate of drug release. For example, SAIB may be formulated with one or more solvents, including but not limited to, nonhydroxylic solvents such as benzyl benzoate, N-methyl-2-pyrrolidone (NMP), dimethylsulfoxide (DMSO), or mixtures thereof. In certain embodiments, it may be desirable to use a solvent such as ethanol, methanol, or glycerol. Where the formulation is to be administered as a spray, a propellant may be added. The solvent can be added to SAIB in a ratio of from about 5% to about 50% solvent.

The angiogenic factor, e.g., in lyophilized to dry powder form, may then be added to the SAIB/solvent mixture, and mixed to homogeneity. The resulting mixture can be administered by injection into the pericardial space. A small incision is made in the pericardium, e.g., by penetration with a needle. The needle is attached to a syringe containing the depot. The depot is injected into the pericardial space and the pericardium may be sewn up or closed with adhesive. Drug is supplied from the depot into the pericardial space, from which it contacts and enters cardiac tissue.

The same method may be used to deliver an anti-arrhythmic.

Alternatively, the depot is sprayed from a needle penetrating the pericardium, directly onto cardiac tissue. A suitable propellant system may be selected from any commonly available system, such as a compressed inert gas, a pump-pressurized system, or a freon propellant system. The depot adheres to the cardiac tissue, and drug passes directly into the tissue. This direct spraying method may be particularly useful for delivering an anti-arrhythmic, directly after heart surgery, but prior to closing up the patient. The anti-arrhythmic would prevent arrhythmia that would otherwise necessitate an expensive hospital stay.

Drug Delivery Devices

Drug Delivery Devices Generally

A drug can be administered into the pericardial fluid using any of a number of delivery systems, including sustained release devices. In some embodiments, the drug delivery system will comprise a catheter operably attached to a sustained release drug delivery device. A proximal end of the catheter is operably attached to a sustained release drug delivery device; and a distal end of the catheter may be adapted for transpericardial delivery, or may be adapted for intrapericardial delivery. In other embodiments, the drug delivery device is a depot.

In general, the drug delivery devices suitable for use in the invention comprise a drug reservoir for retaining a drug formulation or alternatively some substrate or matrix which can retain drug (e.g., a polymer; a viscous non-polymer compound, e.g., as described in U.S. Pat. No. 5,747,058 and U.S. application Ser. No. 09/385,107; a binding solid, etc). Sustained release devices include implantable devices and devices which are not implanted in the body of the subject.

The delivery device is generally adapted for delivery of a drug over extended periods of time. Such delivery devices may be adapted for administration of a drug for several hours (e.g. greater than 12 hours), days (e.g. greater than 7 days), weeks (e.g. greater than 4 weeks) months (e.g. greater than three months) or years.

Release of drug from the device can be accomplished in any of a variety of ways according to methods well known in the art, e.g., by incorporation of drug into a polymer that provides for sustained diffusion of drug from within the polymer, incorporation of drug in a biodegradable polymer, providing for delivery of drug from an osmotically-driven device, etc. Where the drug delivery device comprises a drug delivery catheter, drug can be delivered through the drug delivery catheter to the pericardium or myocardial tissue as a result of capillary action, as a result of pressure generated from the drug release device, by diffusion, by electrodiffusion or by electroosmosis through the device and/or the catheter.

The drug delivery device must be capable of carrying the drug formulation in such quantities and concentration as therapeutically required, and must provide sufficient protection to the formulation from attack by body processes for the duration of implantation (if implanted) and delivery. The exterior is thus preferably made of a material that has properties to diminish the risk of leakage, cracking, breakage, or distortion so as to prevent expelling of its contents in an uncontrolled manner under stresses it would be subjected to during use, e.g., due to physical forces exerted upon the drug release device as a result of movement by the subject or physical forces associated with pressure generated within the reservoir associated with drug delivery. The drug reservoir or other means for holding or containing the drug must also be of such material as to avoid unintended reactions with the active agent formulation, and is preferably biocompatible. Suitable materials for the reservoir or drug holding means may comprise a non-reactive polymer or a biocompatible metal or alloy. Exemplary polymers include, but are not necessarily limited to, biocompatible polymers, including biostable polymers and biodegradable polymers. Exemplary biostable polymers include, silicone, polyurethane, polyether urethane, polyether urethane urea, polyamide, polyacetal, polyester, poly ethylene-chlorotrifluoro-ethylene, polytetrafluoroethylene (PTFE or “Teflon™”), styrene butadiene rubber, polyethylene, polypropylene, polyphenylene oxide-polystyrene, poly-a-chloro-p-xylene, polymethylpentene, polysulfone and other related biostable polymers. Exemplary biodegradable polymers include, but are not necessarily limited to, polyanhydrides, cyclodextrans, polylactic-glycolic acid, polycaprolactone, polyorthoesters, n-vinyl alcohol, polyethylene oxide/polyethylene terephthalate, polyglycolic acid, polylactic acid and copolymers thereof, and other related bioabsorbable polymers.

Drug release devices suitable for use in the invention may be based on any of a variety of modes of operation. For example, the drug release device can be based upon a diffusive system, a convective system, or an erodible system (e.g., an erosion-based system). For example, the drug release device can be an osmotic pump, an electroosmotic pump, an electrochemical pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

A drug delivery device of the invention may release drug in a range of rates of from about 0.01 microgram/hr to about 500 microgram /hr, and which can be delivered at a volume rate of from about 0.01 microliter/day to about 100 microliter/day, e.g. 0.2 microliter/day to about 5 microliter/day. In particular embodiments, the volume/time delivery rate is substantially constant (e.g., delivery is generally at a rate of about 5% to 10% of the cited volume over the cited time period.

The drug delivery device can be implanted at any suitable implantation site using methods and devices well known in the art. An implantation site is a site within the body of a subject at which a drug delivery device is introduced and positioned. Implantation sites include, but are not necessarily limited to myocardial, within the wall of a vessel, and may also be subdermal, subcutaneous, intramuscular etc. Delivery of drug from a drug delivery device at an implantation site that is distant from the myocardium is generally accomplished by providing the drug delivery device with a catheter.

Pumps

Drug release devices based upon a mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. The DUROS™ osmotic pump is particularly suitable (see, e.g., WO 97/27840 and U.S. Pat. Nos. 5,985,305 and 5,728,396, hereby incorporated by reference).

Depots

The drug delivery device can be a depot. Depots are injectable drug delivery devices that may comprise polymeric and/or non-polymeric materials, and are provided in liquid, or semi-solid forms that release drug over time.

Exemplary non-polymeric materials useful in making a depot dosage form include, but are not necessarily limited to, those described in U.S. Pat. Nos. 6,051,558; 5,747,058; and 5,968,542, e.g. a non-polymeric material having a viscosity of at least 5000 cP at 37° C., for example, SAIB.

Suitable polymeric materials include, but are not limited to, polyanhydrides; polyesters such as polyglycolides and polylactide-co-glycolides; polyamino acids such as polylysine; polymers and co-polymers of polyethylene oxide; acrylic terminated polyethylene oxide; polyamides; polycaprolactone, polyurethanes; polyorthoesters; polyacrylonitriles; and polyphosphazenes. See, e.g., U.S. Pat. Nos. 4,891,225; 4,906,474; 4,767,628; and 4,530,840. Degradable materials of biological origin include, but are not limited to, cross-linked gelatin; and hyaluronic acid (e.g., U.S. Pat. No. 4,767,628). A depot may also be provided in the form of a biodegradable hydrogel. See, e.g., U.S. Pat. No. 5,149,543. Depots also include materials that exist in one physical state outside the body, and assume a different physical state when introduced into the body. Examples include liquid materials that form solids when placed within an individual, with or without addition of a catalyst. See, e.g., U.S. Pat. No. 4,938,763. A number of factors well known to those familiar with the art will have an effect on depot release kinetics and should be considered in designing an effective formulation. For example a smaller injection will give a depot with a larger surface-to-volume ratio than a depot resulting from a larger injection. For example, one formulation tested in vitro may have a burst of over 50% when evaluated at a 100 mg depot size and less than 25% when evaluated at a 1000 mg depot size.

Polymer Rods

In certain embodiments, the drug delivery device may be a biodegradable monolithic rod. An experimental example of one such embodiment is a monolithic rod prepared by melt extrusion of a sodium cromoglycate-polymer mixture using, as the polymer poly (d1-lactide-co-glycolide) or poly (caprolactone). Other polymers that may be used are well known. The extruded rod is implanted in the subject using standard surgical techniques under local anesthetic. In certain embodiments, the drug delivery device may be a coaxial rod, in which there is drug in the core as well as the sheath. The polymer used to make the rod could be any suitable polymer, which would be easily determinable by one of skill in the art, for example polyhydroxy acids, such as poly(lactide)s, poly(glycolide)s, poly(lactide-co-glycolide)s, poly(lactic acid)s, poly(glycolic acid)s, and poly(lactic acid-co-glycolic acid)s, polyanhydrides, polyorthoesters, polyetheresters, polycaprolactone, polyesteramides, polyphosphazines, polycarbonates, polyamides, and copolymers and blends thereof. A preferred material is polycaprolactone. The extruded rod is implanted in the subject using standard surgical techniques under local anesthetic. A biodegradable monolithic rod may also be used. An experimental example of such an embodiment is one in which a monolithic rod is prepared by melt extrusion using a Tinius Olsen extruder, wherein the rod contains 20% statin by weight within a polymer of 65:35 poly (DL-lactide-co-glycolide).

Alternatively, the drug delivery device can be a dispersion system, e.g., a suspension or an emulsion. Suspensions are solid particles ranging in size from a few nanometers to hundreds of micrometers, dispersed in a liquid medium using a suspending agent. Solid particles include microspheres, microcapsules, and nanospheres. Emulsions are dispersions of one liquid in another, stabilized by an interfacial film of emulsifiers such as surfactants and lipids. Emulsion formulations include water in oil and oil in water emulsions, multiple emulsions, microemulsions, microdroplets, and liposome emulsions.

Drugs for Treating Cardiac Conditions

Suitable drugs include, but not limited to, growth factors, angiogenic agents, calcium channel blockers, antihypertensive agents, inotropic agents, antiatherogenic agents, anti-coagulants, beta-blockers, anti-arrhythmia agents, vasodilators, thrombolytic agents, cardiac glycosides, anti-inflammatory agents, antibiotics, antiviral agents, antifungal agents, agents that inhibit protozoan infections, antineoplastic agents, and steroids.

Angiogenic factors are as described above.

Calcium channel blockers include, but are not limited to, dihydropyridines such as nifedipine, nicardipine, nimodipine, and the like; benzothiazepines such as dilitazem; phenylalkylamines such as verapamil; diarylaminopropylamine ethers such as bepridil; and benzimidole-substituted tetralines such as mibefradil.

Antihypertensive agents include, but are not limited to, diuretics, including thiazides such as hydroclorothiazide, furosemide, spironolactone, triamterene, and amiloride; antiadrenergic agents, including clonidine, guanabenz, guanfacine, methyldopa, trimethaphan, reserpine, guanethidine, guanadrel, phentolamine, phenoxybenzamine, prazosin, terazosin, doxazosin, propanolol, methoprolol, nadolol, atenolol, timolol, betaxolol, carteolol, pindolol, acebutolol, labetalol; vasodilators, including hydralizine, minoxidil, diazoxide, nitroprusside; and angiotensin converting enzyme inhibitors, including captopril, benazepril, enalapril, enalaprilat, fosinopril, lisinopril, quinapril, ramipril; angiotensin receptor antagonists, such as losartan; and calcium channel antagonists, including nifedine, amlodipine, felodipine XL, isadipine, nicardipine, benzothiazepines (e.g., diltiazem), and phenylalkylamines (e.g. verapamil).

Anti-coagulants include, but are not limited to, heparin; warfarin; hirudin; tick anti-coagulant peptide; low molecular weight heparins such as enoxaparin, dalteparin, and ardeparin; ticlopidine; danaparoid; argatroban; abciximab; and tirofiban.

Anti-arrhythmic drugs may be local anesthetics, beta-receptor blockers, prolongers of action potential duration or calcium antagonism. Antiarrhythmic agents include, but are not necessarily limited to, sodium channel blockers (e.g., lidocaine, sotatol, procainamide, encainide, flecanide, and the like), beta adrenergic blockers (e.g., propranolol, dopamine-beta-hydroxylase inhibitors), prolongers of the action potential duration (e.g., amiodarone), and calcium channel blockers (e.g., verpamil, diltiazem, nickel chloride, and the like). Delivery of cardiac depressants (e.g., lidocaine), cardiac stimulants (e.g., isoproterenol, dopamine, norepinephrine, etc.), and combinations of multiple cardiac agents (e.g., digoxin/quinidine to treat atrial fibrillation) is also of interest.

Agents to treat congestive heart failure, include, but are not limited to, a cardiac glycoside, a loop diuretic, a thiazide diuretic, a potassium ion sparing diuretic, an angiotensin converting enzyme inhibitor, an angiotension receptor antagonist, a nitrovasodilator, a phosphodiesterase inhibitor, a direct vasodilator, an alpha₁-adrenergic receptor antagonist, a calcium channel blocker, and a sympathomimetic agent.

Thrombolytic agents include, but are not limited to, urokinase plasminogen activator, urokinase, streptokinase, inhibitors of alpha2-plasmin inhibitor, inhibitors of plasminogen activator inhibitor-1, angiotensin converting enzyme (ACE) inhibitors, spironolactone, tissue plasminogen activator (tPA), inhibitors of interleukin 1 beta converting enzyme, anti-thrombin III, and the like.

Agents suitable for treating cardiomyopathies include, but are not limited to, dopamine, epinephrine, norepinephrine, and phenylephrine.

Anti-inflarmatory agents include, but are not limited to, any known non-steroidal anti-inflammatory agent, and any known steroidal anti-inflammatory agent.

Antimicrobial agents include antibiotics (e.g. antibacterial), antiviral agents, antifungal agents, and anti-protozoan agents.

Antineoplastic agents include, but are not limited to, those which are suitable for treating cardiac tumors (e.g., myxoma, lipoma, papillary fibroelastoma, rhabdomyoma, fibroma, hemangioma, teratoma, mesothelioma of the AV node, sarcomas, lymphoma, and tumors that metastasize to the heart) including cancer chemotherapeutic agents, a variety of which are well known in the art.

Dosages

Suitable dosages may depend on several factors, including the potency of the drug being administered, the desired therapeutic effect, the duration of administration, etc. Those skilled in the art can readily determine appropriate dosages. In general, dosages (expressed as amount of drug per kg body weight of the subject) will vary from about 0.1 micrograms/kg to about 500 mg/kg, from about 1 micrograms/kg to about 100 mg/kg, from about 10 micrograms/kg to about 50 mg/kg, from about 50 micrograms/kg to about 25 mg/kg, from about 100 micrograms/kg to about 10 mg/kg, or from about 1 mg/kg to about 5 mg/kg. These dosages are total dosages per administration.

Formulations

In general, drugs are prepared in a pharmaceutically acceptable composition for delivery to a subject. Pharmaceutically acceptable carriers for use with a drug may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, and microparticles, including saline and buffered media. Other vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

In general, the pharmaceutical compositions are prepared in various liquid forms. Pharmaceutical grade organic or inorganic carriers and/or diluents suitable for cardiac delivery can be used to make up compositions comprising the therapeutically-active compounds. Diluents known to the art include aqueous media, vegetable and animal oils and fats. Stabilizing agents, wetting and emulsifying agents, and salts for varying the osmotic pressure or buffers for securing an adequate pH value can be used as auxiliary agents. Preservatives and other additives may also be present such as, for example, antimicrobials, antioxidants, chelating agents, and inert gases and the like.

Methods of Treatment

The present invention provides methods of treating an individual having a cardiac pathology comprising administering a pharmaceutically active agent to the individual using a continuous delivery method of the invention. Generally the drug is delivered from a sustained-release dosage form implanted in the myocardial or vascular tissue.

In one exemplary embodiment FGF is delivered to myocardial tissue using an implanted osmotic pump fitted with a catheter. FGF is formulated with heparin and saline to a concentration of 1% and loaded into an osmotic pump. Release rate from the pump is about 0.5 μg/hr. The pump is implanted at a site outside the myocardium, preferably subcutaneously, in the chest area, under the arm. The catheter is threaded through the chest wall to the heart where the distal end is implanted into the myocardial tissue and fixed in place using sutures.

In another embodiment FGF is delivered to pericardium or myocardial tissue using a depot comprising sucrose acetate isobutyrate (SAIB). The depot is implanted by injection in the myocardial tissue where it releases FGF, stimulating angiogenesis. FGF is released at a rate of up to 1 μL/hr/Kg.

In exemplary embodiments, SAIB may be formulated with one or more solvents which may be nonhydroxylic or hydroxylic and which may be used alone or in combination. Examples of solvents include benzyl benzoate, N-methyl-2-pyrrolidone (NMP), dimethylsulfoxide (DMSO), benzoic acid, ethyl lactate, propylene carbonate, glycofurol, glycerol, Miglyol 810, ethanol, or mixtures thereof. Where the formulation is to be administered as a spray, a propellant may be added. The solvent can be added to SAIB in a ratio of from about 5 wt % to about 65 wt % solvent, usually 50 wt % or less. The angiogenic factor, e.g., in lyophilized or dry powder form, may then be added to the SAIB/solvent mixture, and mixed to achieve homogeneity. Mixing may be accomplished by any acceptable means including passing between syringes fitted with needles or passing through a roll mill or mixing with a homogenizer. The resulting mixture (the depot) can be administered by injection into the pericardium or myocardial tissue using a syringe fitted with a 25-26 gauge needle. An appropriate implantation site for angiogenic factors is within ischemic tissue. Antiarrhythmic agents, may be implanted anywhere within the myocardium. Drug is released from the depot into the myocardial tissue, stimulating angiogenesis.

In another embodiment, the depot, such as a SAIB depot formulated with a solvent and a drug, is sprayed directly onto cardiac tissue. A suitable propellant system may be selected from any commonly available system, such as a compressed inert gas, a pump-pressurized system, or a chlorofluorocarbon (e.g., Freon propellant system. The depot adheres to the cardiac tissue, and drug passes directly into the tissue. Such an embodiment may be of particular use for applying an anti-arrhythmic agent, such as a beta-blocker, directly to the surface of the heart, following heart surgery. Such a treatment would reduce the incidence of post-operative arrhythmia, thereby reducing hospitalization time and cost.

In another embodiment, the formulation may be in the form of a biodegradable rod made of a polymer with an appropriate drug such as VEGF. An experimental example of one such embodiment is a biodegradable rod made of 65:35 poly (dl-lactide-co-glycolide) to which 5% of PEG 1000 has been added as a porasigen. The extruded rod is a hollow tube to which is added VEGF along with excipients and protein stabilizers. The ends of the rod are sealed. This formulation demonstrated about 50% release of VEGF over a 25-day period. A similarly prepared rod with as an extruded hollow tube made of caprolactone demonstrated VEGF release over a 30-day period.

In another embodiment the formulation may be in the form of a depot comprising microspheres. For example, FGF loaded microspheres may be prepared using poly (dL-lactide) (DL-PL) as the excipient (see Example 8).

The present invention also provides methods where the drug is delivered from a sustained-release dosage form implanted in the pericardial space.

In one exemplary embodiment FGF is delivered to pericardium using an implanted osmotic pump fitted with a catheter. FGF is formulated as described herein. The pump is implanted at a site outside the heart, preferably subcutaneously, in the chest area, under the arm. The catheter is threaded through the chest wall where the distal end is implanted into the pericardium and fixed in place using sutures.

In another embodiment FGF is delivered to pericardium using a depot comprising sucrose acetate isobutyrate (SAIB). The depot is implanted by injection in the pericardium myocardial tissue where it releases FGF, stimulating angiogenesis. FGF is released into the pericardial space, contacting the cardiac tissue, at a rate of up to 1 μL/hr/Kg.

In exemplary embodiments, SAIB may be formulated with one or more solvents which may be nonhydroxylic or hydroxylic and which may be used alone or in combination. Examples of solvents include benzyl benzoate, N-methyl-2-pyrrolidone (NMP), dimethylsulfoxide (DMSO), benzoic acid, ethyl lactate, propylene carbonate, glycofurol, glycerol, Miglyol 810, ethanol, or mixtures thereof. Where the formulation is to be administered as a spray, a propellant may be added. The solvent can be added to SAID in a ratio of from about 5 wt % to about 65 wt % solvent, usually 50 wt % or less. The angiogenic factor, e.g., in lyophilized or dry powder form, may then be added to the SAID/solvent mixture, and mixed to achieve homogeneity. Mixing may be accomplished by any acceptable means including passing between syringes fitted with needles or passing through a roll mill or mixing with a homogenizer. The resulting mixture (the depot) can be administered by injection into the pericardium using a syringe fitted with a 25-26 gauge needle. Drug is released from the depot into the pericardium, stimulating angiogenesis.

In another embodiment, the formulation may be in the form of a biodegradable rod made of a polymer or a depot comprising microspheres, as above, implanted into the pericardial sac.

Subjects Suitable for Treatment

Subjects suitable for treatment using the methods of the present invention include individuals having a condition that is treatable by increasing angiogenesis in cardiac tissue. Such conditions include, but are not limited to, (1) chronic stable angina; (2) unstable angina; (3) acute myocardial infarction; (4) hibernating myocardium; (5) stunned myocardium; (6) limitation of ventricular remodeling in post myocardial infarction and subsequent risk of congestive heart failure; (7) prophylaxis of recurrent myocardial infarction; (8) prevention of sudden death following myocardial infarction; (9) vasospastic angina; (10) congestive heart failure-systolic-seen in association with 1-6 above; (11) congestive heart failure-diastolic-seen in association with 1-10 above and 12-15 below; (12) microvascular angina seen in association with 1-11 above and 15 and 16 below; (13) silent ischemia seen in association with 1-12 above and 15 and 16 below; (14) reduction of ventricular ectopic activity seen in association with 1-13 above and 15 below; (15) any or all of the above 1-14 states of ischemic myocardium associated with hypertensive heart disease and impaired coronary vasodilator reserve; (16) control of blood pressure in the treatment of hypertensive crisis, perioperative hypertension, uncomplicated essential hypertension and secondary hypertension; (17) regression of left ventricular hypertrophy seen in association with 15 and 16 above; (18) prevention and or regression of epicardial coronary arteriosclerosis seen in 1-17 above; (19) prevention of restenosis post angioplasty; (20) prevention and/or amelioration of free radical mediated reperfusion injury in association with 1-19 above; (21) use of the combination in the prevention of myocardial injury during cardioplegic arrest during coronary bypass or other open heart surgery i.e. use of the combination as a cardioplegic solution; (22) post transplant cardiomyopathy; (23) renovascular ischemia; (24) cerebrovascular ischemia (TIA) and stroke); (25) pulmonary hypertension; and (26) peripheral vascular disease (claudication), and (27) individuals suffering an ischemic heart disease; (28) arrhythmia; (29) a cardiomyopathy; (30) coronary angioplasty restenosis; (31) cardiac inflammation; (32) myocardial infarction; (33) atherosclerosis; (34) thrombosis; (35) a cardiac condition related to hypertension; (36) cardiac tamponade; (37) pericardial effusion; and (38) a cardiac neoplasm.

Ischemic disease and attendant syndromes include, but are not limited to, myocardial infarction; stable and unstable angina; coronary artery restenosis following percutaneous transluminal coronary angioplasty; and reperfusion injury.

Cardiomyopathies include, but are not limited to, cardiomyopathies caused by or associated with ischemic syndromes; cardiotoxins such as alcohol, and chemotherapeutic agents such as adriamycin; microbial infections of cardiac tissue, (or deleterious effects of microbial infections of other tissues (e.g., toxin production)), due to any microbial agent including viruses, e.g. cytomegalovirus, human immunodeficiency virus, echovirus, influenza virus, adenovirus; bacteria, including, but not limited to, Mycobacterium tuberculosis, meningococci, spirochetes, viridans Streptococci, (e.g., S. sanguis, S. oralis, S. salivarus. S. mutans), Enterococci, Staphylococci (e.g., S. aureus, S. epidermidis), Haemophilus parainfluenzae, Haemophilus aphrophilus, Eikenella corrdens, Kingella kingae, Actinobacillus actinomycetemcomitans, Cardiobacterium hominus; protozoans, such as Trypanosoma cruzi; and fungi, including, but not limited to, Candida parapsilosis, Candida albicans, and Candida tropicalis; hypertension; metabolic disorders, including, but not limited to, uremia, and glycogen storage disease; radiation; neuromuscular disease (e.g., Duchene's' muscular dystrophy); infiltrative diseases (e.g., sarcoidosis, hemochromatosis, amyloidosis); trauma; and idiopathic causes.

Inflammatory conditions include, but are not limited to, myocarditis, pericarditis, endocarditis, immune cardiac rejection, and conditions resulting from idiopathic, autoimmune, or connective tissue diseases.

Infections of cardiac tissues may be bacterial, viral, fungal, or parasitic (e.g., protozoan) in origin (see above for non-limiting list of microbial infectious agents).

### EXAMPLES

The following examples are put forth so as to provide those of ordinary skill in the art with a complete disclosure and description of how to make and use the present invention, and are not intended to limit the scope of what the inventors regard as their invention. Unless indicated otherwise, parts are parts by weight, molecular weight is weight average molecular weight, temperature is in degrees Celsius, and pressure is at or near atmospheric.

### Example 1

FGF Delivered to Myocardial Tissue from an Osmotic Pump with a Catheter

A DUROS™ or ALZET™ osmotic pump is used to deliver a formulation containing FGF to the heart. A catheter is used to deliver the drug formulation from the pump to the target site. The pump is implanted at a site outside the myocardium, preferably subcutaneously, in the chest area, under the arm. The catheter is threaded through the chest wall to the heart where the distal end is implanted into the myocardial tissue and fixed in place using sutures.

The formulation consists of 1% FGF and 0.033% heparin in PBS (USP) buffer. The formulation is prepared by dissolving Fibroblast Growth Factor (Sigma Chemical Co.) and heparin (Sigma Chemical Company) in PBS (USP) to form a solution containing 1% FGF and 0.033% of heparin. An osmotic pump is then filled with the formulation with a syringe under aseptic conditions. A DUROS™ pump may be used, having a drug capacity of 150 microliters. The release rate of formulation from the pump is adjustable, but is generally about 1.5 to 5 microliters/day, but may be up to 2 ml/day. The FGF formulation is delivered from the pump into the myocardial tissue, from where it contacts and enters the cardiac cells, stimulating angiogenesis.

### Example 1A

FGF Delivered to the Pericardial Space from an Osmotic Pump with a Catheter

An osmotic pump may be used to deliver a formulation containing FGF to the pericardial space of the heart. The pump is implanted at a site outside the heart, preferably subcutaneously, in the chest area, under the arm. The catheter is threaded through the chest wall to the heart where the distal end is implanted through an incision in the pericardial membrane into the pericardium or myocardial tissue and fixed in place using sutures.

The formulation consists of 1% FGF and 0.033% heparin in PBS (USP) buffer. The formulation is prepared by dissolving Fibroblast Growth Factor (Sigma Chemical Co.) and heparin (Sigma Chemical Company) in PBS (USP) to form a solution containing 1% FGF and 0.033% of heparin. An osmotic pump is then filled with the formulation with a syringe under aseptic conditions. A DUROS™ pump may be used, having a drug capacity of 150 microliters. The release rate of formulation from the pump is adjustable, but is generally about 1.5 to 5 microliters/day, but may be up to 2 ml/day. The FGF formulation is delivered from the pump into the pericardial space, from where it contacts and enters the cardiac cells, stimulating angiogenesis.

### Example 2

FGF Delivered from a SAIB Depot to Myocardial Tissue or to the Pericardium or Sprayed Directly onto the Heart Surface

In this embodiment FGF is delivered from a depot comprising sucrose acetate isobutyrate (SAIB). A formulation is prepared by mixing SAIB (Eastman Chemical Co.) and benzyl benzoate (Aldrich Chemical Co.) and ploy(DL-lactide-co-glycolide) (DL-PLG) or DL-poly (lactide) (DLPL) in a ratio of 83:12:5 (weight basis) and stirring until a homogeneous mixture is achieved. 10 μg of human, recombinant Fibroblast Growth Factor (FGF) (Sigma Chemical Co.) is added to 500 μL of the SAIB:benzyl benzoate:DLPLG formulation.

The final depot formulation is prepared by passing the mixture repeatedly between a pair of 5 ml syringes equipped with needles. Multiple passes are performed until a homogeneous suspension is achieved. The final concentration of FGF in the depot is 0.002 μg/μL.

To determine, in vitro, the release of FGF from the formulation, 500 μL of the depot is placed in 750 μL of dissolution buffer (PBS, 0.01 M, pH 7.4 with sodium azide) in a 1.5 mL Eppendorf microcentrifuge tube. The formulations are incubated at 37° C. with no agitation. The entire dissolution buffer is removed and replaced with fresh buffer at the desired sampling times (0.25, 0.5, 1, 2, 3, 4, 5, 6, 24 hr and daily thereafter). The samples are assayed for protein concentration by ELISA. The release rate of drug from this depot is about 0.3 μg per day.

The FGF depot so prepared may be injected directly into myocardial tissue, or placed in the pericardial sack by injection through the pericardium using a large gauge needle, from where it slowly releases FGF.

Alternatively, the SAIB-FGF formulation may be sprayed, from a compressed gas or pump sprayer, directly onto the surface of the heart, where it will stick, and release FGF over time.

### Example 2A

Sotalol Delivered from SAIB Depot

In this embodiment sotalol (an anti-arrhythmic) is delivered from a depot comprising sucrose acetate isobutyrate (SAIB). A formulation is prepared by mixing SAIB (Eastman Chemical Co.) and benzyl benzoate (Aldrich Chemical Co.) and ploy (DL-lactide-co-glycolide) (DL-PLG) or DL-poly (lactide) (DLPL) in a ratio of 83:12:5 (weight basis) and stirring until a homogeneous mixture is achieved. 10 μg of sotalol is added to 500 μL of the SAIB:benzyl benzoate:DLPLG formulation.

The final depot formulation is prepared by passing the mixture repeatedly between a pair of 5 ml syringes equipped with needles. Multiple passes are performed until a solution is achieved. The final concentration of sotalol in the depot is 0.02 μg/ul.

To determine, in vitro, the release of propranolol from the formulation, 500 μL of the depot is placed in 750 μL of dissolution buffer (PBS, 0.01 M, pH 7.4 with sodium azide) in a 1.5 mL Eppendorf microcentrifuge tube. The formulations are incubated at 37° C. with no agitation. The entire dissolution buffer is removed and replaced with fresh buffer at the desired sampling times (0.25, 0.5, 1, 2, 3, 4, 5, 6, 24 hr and daily thereafter). The samples are assayed for protein concentration by ELISA. The release rate of drug from this depot is about 0.3 μg per day.

The propranolol depot so prepared may be placed in the pericardial sack by injection through the pericardium using a large gauge needle, from where it slowly releases propranolol. Alternatively, the SAIB-propranolol formulation may be sprayed, from a compressed gas or pump sprayer, directly onto the surface of the heart, where it will stick, and release propranolol over time.

### Example 3

FGF Delivered from a Biodegradable Rod

In this embodiment FGF is delivered from a biodegradable rod. The monolithic rod dosage form is formulated and prepared as an extended hollow rod. To prepare this formulation a hollow tube of 65:35 poly(dl-lactide-co-glycolide) to which 5% of PEG-1000 is added as a porasigen is extruded on a Randcastle extruder using a standard tubing dye. The resulting hollow rod is cut to the desired length. The rod is filled with a preparation of 25 wt % FGF and 75 wt % PEG-400 to serve as a excipient and stabilizer for the protein. The rods are assayed for release of FGF by placing in 40 mL of dissolution buffer (HEPES) in a 120 or 240 mL amber bottle at 37° C. with no agitation. After incubation for 1 hr, 5 mL of buffer is removed for analysis and replaced with fresh buffer. Samples are removed for analysis daily for one week and weekly thereafter. Analysis of the samples for FGF content is accomplished by ELISA. The formulation showed a lag in release for 2 days and then released about 3% of the loading/day for 30 days. The formulation shows a lag in release for two days and then released approximately 3% of the load per day for 30 days.

### Example 4

FGF Delivered from a Depot Comprising Microspheres

FGF-loaded microspheres are prepared using poly (dL-lactide) (DL-PL) as the excipient. The inherent viscosity of the DL-PL in chloroform (30° C.) is 0.65 dL/g. The dispersed phase (DP) is a solution containing 10 g of DL-PL and 25 μg of FGF dissolved in 166.67 g of dichloromethane (DCM). The continuous phase (CP) is prepared by dissolving 5.26 g of DCM in a 6 wt % solution of poly(vinyl alcohol). The extraction phase consists of deionized water and is calculated to provide 90% extraction of the DCM from the microspheres. The amount of required extraction phase (9342.9 g) is transferred to a 12-L spherical reaction flask fitted with a lid, a vacuum adapter connected to a water aspirator and an overhead stirrer fitted with a 6-blade impeller. The stirrer is set to approximately 510 rpm. The CP is transferred to a 1-L cylindrical reaction flask fitted with a lid and an overhead stirrer fitted with a 6-blade impeller. The CP stirrer is set to approximately 650 rpm. The DP is added to the CP with stirring to form the primary emulsion. After 5 minutes, the emulsion is transferred to the 12-L reaction flask containing the EP to initiate extraction of the DCM thereby forming microspheres. After about 10 minutes, the flask is closed and evacuated using the water aspirator. The pressure inside the flask is gradually reduced from about 35 mm Hg below atmospheric to about 584 mm Hg below atmospheric over about six hours. After about 24 hr, the microspheres are collected on a flitted glass funnel, washed with deionized water and vacuum dried to yield a free flowing powder. The microspheres have a diameter from about 10 μm to about 150 μm. The microspheres are assayed to determine FGF content by dissolving in acetonitrile, diluting with PBS (0.01 M, pH 7.4 with sodium azide), and assaying by HPLC. To determine the release of FGF from the microspheres, a known amount of microspheres is placed into 250 mL of dissolution buffer (PBS, 0.01 M, pH 7.4 with sodium azide) prewarmed to 37° C. in a 250-mL round bottom flask. The flasks are agitated at 125 rpm in an orbital shaker. Samples are removed at 0.25, 0.5, 1, 2, 3, 4, 5, 6, and 24 hr and daily thereafter. The samples are assayed for FGF by HPLC. The formulation shows a burst of drug of 25% in the first day and releases the balance of drug in first-order kinetics over 21 days. The formulation shows a cumulative burst of drug of 25% in the first day and releases the balance of the drug at a rate characterized by first order kinetics over 21 days.

The microspheres so prepared may be placed in the pericardial sack by injection through the pericardium using a large gauge needle, from where they slowly release FGF.

### Example 4A

Propranolol Delivered from a Depot Comprising Microspheres

Propranolol (an anti-arrhythmic)—loaded microspheres are prepared using poly (dL-lactide) (DL-PL) as the propranolol, exactly as above, for FGF. The microspheres are assayed to determine propranolol content by dissolving in acetonitrile, diluting with PBS (0.01 M, pH 7.4 with sodium azide), and assaying by HPLC. To determine the release of propranolol from the microspheres, a known amount of microspheres is placed into 250 mL of dissolution buffer (PBS, 0.01 M, pH 7.4 with sodium azide) prewarmed to 37° C. in a 250-mL round bottom flask. The flasks are agitated at 125 rpm in an orbital shaker. Samples are removed at 0.25, 0.5, 1, 2, 3, 4, 5, 6, and 24 hr and daily thereafter. The samples are assayed for propranolol by HPLC. The microspheres so prepared may be placed in the pericardial sack by injection through the pericardium using a large gauge needle, from where they slowly release propranolol.

### Example 5

Bolus Injection of Compounds into the Pericarial Space

Immediately after implantation of the pericardial catheter, rats (still under anesthesia) were provided either with a catheter in the right femoral artery essentially as described (Smits et al., 1982). Rats were allowed to recover at least 2 days before experimentation>One hour before start of the experiment, 20 μl pericardial fluid was withdrawn using a Hamilton 1705 (Hamilton Bonaduz, Bonaduz, Switzerland) syringe and 50 μl of saline were injected into pericardial space to check the integrity of the pericardial catheter. Injections of volumes up to 0.2 ml were previously shown to be without hemodynamic effects (Veelken et al., 1990). Blood (0.15-0.25 ml) was collected in a syringe, containing a minimal volume of heparin (Organon Teknika, Boxtel, the Netherlands). Pericardial fluid was diluted 10 times in PBS and the blood was centrifuged for 20 minutes at 3500 rpm to obtain plasma. These samples served as blanks for later analyses. Experiments in which substances were applied intrapericardially were started by a 50 μl bolus injection of the test substances into pericardial space, followed by 20 μl saline to flush the catheter. If substances were applied systemically, experiments were started by a 100 μl bolus injection of the substances and subsequent injection of 300 μl saline into the femoral artery catheter. FITC rat IgG, (10 mg/ml), Texas Red RSA (10 mg/ml), and FITC heparin (1 mg/ml) were dissolved in PBS. Texas Red FGF-2 (20 μg/ml) was dissolved in a 10 mg/ml solution of RSA in PBS.

Pericardial fluid (20 μl) and blood samples were taken at various time points after injection. To substitute withdrawn pericardial fluid, 20 μl of saline was injected into pericardial space immediately after sampling. After every sample, the femoral artery catheter was flushed with 0.3 to 0.4 ml saline and filled with heparinized (5-10 IU/ml) saline. Plasma and pericardial fluid samples were stored at −20° C. until analysis.

Data were standardized for bodyweights. Pharmacokinetic analysis of the data for each animal was conducted using the GPAD (GraphPAD Software, San Diego, Calif.) software package. Data were fitted to the exponential equation Ct=A.e^{-αt}30 B. e^{-βt} of one—(i.e. A is fixed at 0) and two compartment models. Fits were compared using F-tests and data were log transformed for model judgement.

Results

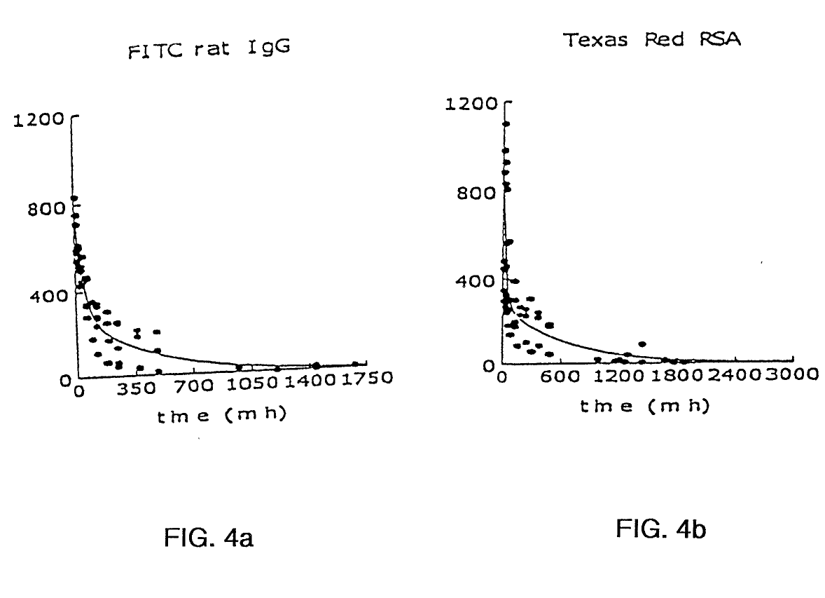

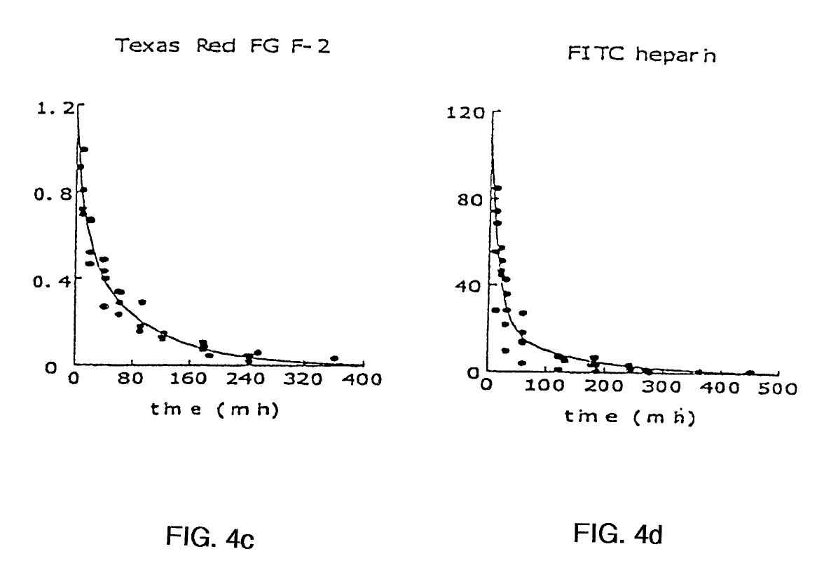

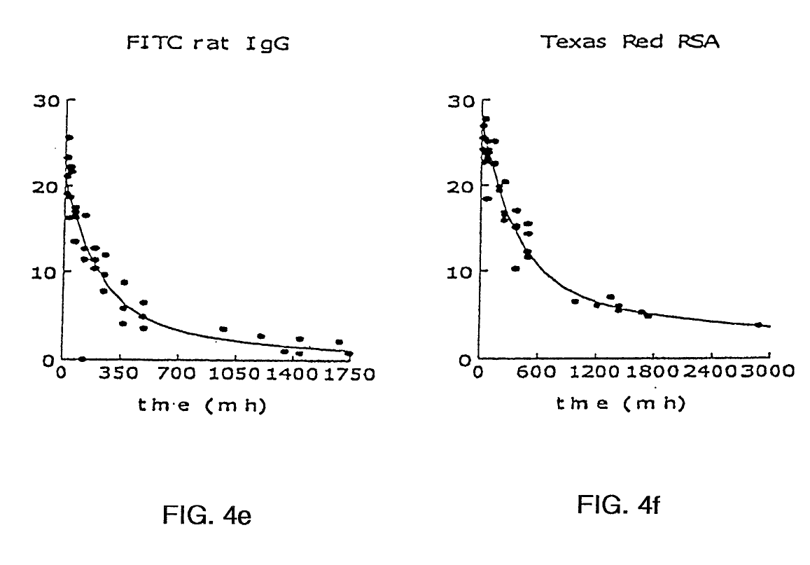

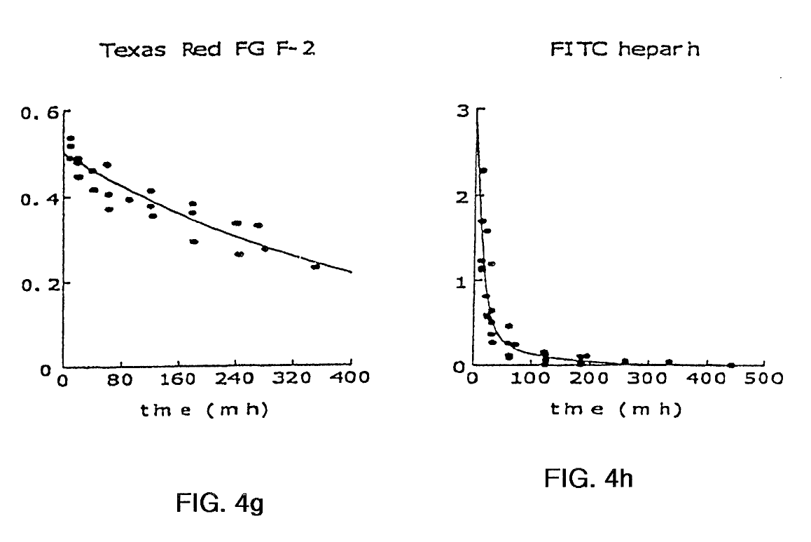

Pericardial fluid concentration-time profiles of intra-pericardially applied and plasma concentration-time profiles of systemically applied FITC rat IgG, Texas Red RSA, Texas Red FGF-2 and FITC heparin are shown in FIG. 4. Pharmacokinetic parameters obtained from the data in FIG. 4, are shown in Table 1.

TABLE 1
Pharmacokinetic parameters of fluorescent macromolecules.

| | A as fraction of | B as fraction of | t_{1/2α}* | t_{1/2β}* | V_{c}** | Cl*** | number |
|---|---|---|---|---|---|---|---|
| | C₀ (see below) | C₀ (see below) | (min) | (min) | (μl/kg) | (μl/min · kg) | of rats |
| Pericardial fluid: | | | | | | | |
| FITC rat IgG | 0.00 | 1.00 | NA | 167 ± 66 | 893 ± 114 | 5.30 ± 1.10 | 6 |
| Texas Red RSA | 0.66 ± 0.11 | 0.34 ± 0.11 | 46.8 ± 14 | 589 ± 133 | 892 ± 207 | 3.72 ± 0.90 | 7 |
| Texas Red FGF-2 | 0.85 ± 0.06 | 0.15 ± 0.06 | 17.3 ± 5.5 | 102 ± 19 | 497 ± 70 | 8.05 ± 0.33 | 4 |
| FITC heparin | 0.82 ± 0.06 | 0.18 ± 0.06 | 12.8 ± 3.9 | 87 ± 18 | 513 ± 86 | 16.8 ± 5.62 | 5 |
| Plasma: | | | | | | | |
| FITC rat IgG | 0.77 ± 0.07 | 0.23 ± 0.07 | 116 ± 18.4 | 657 ± 125 | 46248 ± 3838 | 128 ± 6.38 | 5 |
| Texas Red RSA | 0.59 ± 0.12 | 0.41 ± 0.12 | 89 ± 14.8 | 1132 ± 300 | 34734 ± 1761 | 40.5 ± 2.76 | 5 |
| Texas Red FGF-2 | 0.00 | 1.00 | NA | 338 ± 31 | 39990 ± 923 | 84.2 ± 10.3 | 3 |
| FITC heparin | 0.83 ± 0.06 | 0.17 ± 0.06 | 10.2 ± 2.3 | 79.7 ± 23.2 | 33175 ± 5939 | 1400 ± 303 | 5 |

Parameters were derived by fitting standardized data (FIG. 4) to the equation Ct = A.e^{-αt} + B.e^{-βt} of one (i.e. A is fixed at 0) and two compartment models and are expressed as mean ± SE.
*t_{1/2α} and t_{1/2β} were calculated from 1n2/α and 1n2/β.
**V_{c} = Dose/C₀ is the (initial) central compartment volume (i.e. the volume of the compartment to which the agent is applied); C₀ = A + B is the intercept of the concentration time-curve.
***Cl (clearance) as Dose/AUC (area under the C-t curve).
NA: not applicable (best fit using 1-compartment model).

Pharmacokinetics of the fluorescent macromolecules generally appear to be best described using two-compartment models, indicating (rapid) distribution and (slower) elimination phases for the compounds. However, for intra-pericardially applied FITC rat IgG in pericardial fluid as well as systemically applied Texas Red FGF-2 in plasma, one-compartment models seem to be most appropriate. Calculated (initial) central compartment volumes (V_{c}, representing the volume of the compartment to which the substance is applied) do not vary widely between the substances and range between 33 and 46 ml/kg body weight in plasma and between 0.5 and 0.9 ml/kg bodyweight in pericardial fluid. Pericardial clearances of the macromolecules are 10.6 to 83 fold smaller than plasma clearances. In addition, the difference between the substances regarding their clearances appears to be smaller in pericardial fluid than in plasma.

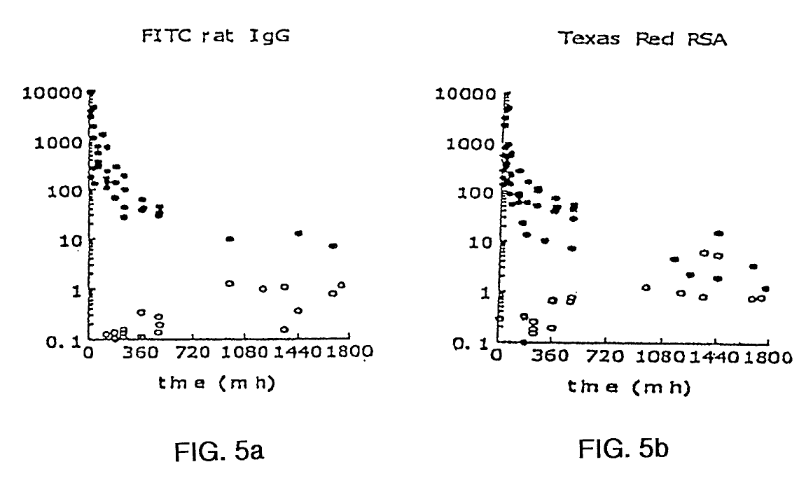

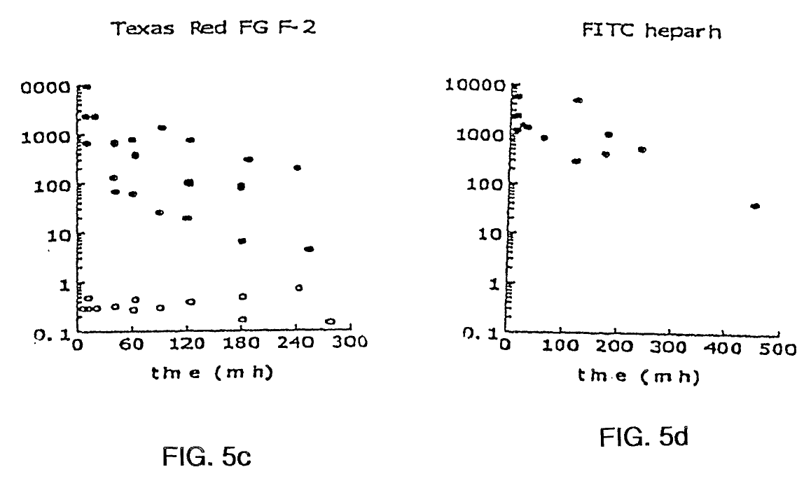

FIG. 5 depicts the ratios of pericardial fluid and plasma concentrations of fluorescent macromolecules after bolus injections into pericardial space or into blood. The data show that upon pericardial bolus injection, pericardial concentrations of the compounds exceed plasma concentrations over a prolonged period of time. On the other hand, following systemic bolus injections, pericardial concentrations are lower than plasma concentrations over an approximately similar period of time, but concentration differences between plasma and pericardial fluid generally are less pronounced than after pericardial application. No data are shown for FITC heparin after intra-arterial injection because pericardial concentrations were below the detection limit.

### Example 6

Infusion of Compounds into the Pericardial Space

Directly following installment of the pericardial catheter, still anesthetized rats were provided with a catheter in the left jugular vein (Kleinjans et al., 1984). Rats were allowed to recover for 2 days, prior to subcutaneous implantation (under ketamine/xylazine anaetesia) of osmotic minipumps (Alzet 2001, Alza Co, Palo Alto, USA). Minipumps, filled with solutions of the substances to be tested, were primed in saline at 37° C. at least 4 hours, prior to connection to the catheter. Before installing pumps, pericardial fluid and orbital sinus blood was sampled, to serve as blanks. 7 days after pump installment, rats were sacrificed by exsanguination under pentobarbitone and pericardial fluid and blood collected. To check for possible loss of substances during infusion, remaining pump contents were analyzed. No significant changes in the concentration of the substances in the infusion fluid were found after 1 week of pumping. Infusion rates of the substances were 10 μg/hour for FITC rat IgG and Texas Red RSA, 20 ng/hour for Texas Red FGF-2, 100 ng/hour for FITC heparin, 684 ng/hour for cortisol and 984 ng/hour for the side-chain modified acid analogue of cortisol. Doses were chosen to achieve concentrations that were readily measurable but without pharmacological effects (risk of bleeding in the case of heparin); similar doses were applied systemically and intrapericardially to be able to make a good comparison between the two routes of administration. Solvent was PBS, except for Texas Red FGF-2 and cortisol which were dissolved in a 10 mg/ml solution of RSA in PBS.

Pericardial fluid and plasma concentrations of substances after 7 days of intrapericardial or intravenous infusion are given in Table 2.

TABLE 2
Pericardial fluid and plasma concentrations of various substances after 7 days of
continuous pericardial or systemic infusion.

| | Intrapericardial Infusion | | | Systemic Infusion | | |
|---|---|---|---|---|---|---|
| | Peric. | | Peric/plasm | | | Peric/plasm |
| | Fluid | Plasma | a ratio (rats) | Peric. Fluid | Plasma | a ratio (rats) |
| FITC | 30.1 ± 10.7 | 3.17 ± 1.13 | 9.83 ± 3.67 (5) | 3.78 ± 1.11 | 5.50 ± 1.51 | 1.36 ± 0.68 (5) |
| rat IgG | | | | | | |
| Texas Red | 39.4 ± 6.93 | 6.35 ± 2.18 | 8.11 ± 2.60 (4) | 2.39 ± 0.58 | 3.07 ± 1.04 | 0.98 ± 0.26 (4) |
| RSA | | | | | | |
| Texas Red | 24.3 ± 13.3 | 4.10 ± 2.36 | 6.85 ± 1.61 (4) | 4.85 ± 2.20 | 4.62 ± 0.47 | 1.01 ± 0.38 (2) |
| FGF-2 | | | | | | |
| FITC- | 42.6 ± 3.4 | n.d. | >30* (4) | n.d. (4) | | |
| heparin | | | | | | |
| Cortisol | 1.59 ± 0.44 | 0.11 ± 0.03 | 14.4 ± 0.55 (2) | Not | | |
| | | | | determined | | |
| Cortisol | 5.42 ± 0.62 | 0.01 ± 0.002 | 420 ± 81 (3) | Not | | |
| carbonic acid | | | | determined | | |

Concentrations are given as fraction of the substance concentration, relative to its concentration in the infusate (infusion rate was 1 μl/hour) and are corrected for bodyweights (i.e bodyweight (kg) × 10000 × measured concentration/infusate concentration).
Data are expressed as mean ± SE. Concentration ratios were calculated for each animal and the number of animals is given in parenthesis.
*No FITC heparin could be detected in plasma, the value of 30 was calculated by dividing the mean pericardial FITC heparin concentration by the detection limit of FITC heparin in plasma.
n.d. Below detection limit.

Based on pilot experiments in which concentrations were determined on a daily basis, as well as on terminal half-lives (Table 1), it is reasonable to assume that after 7 days of infusion, steady state has been reached. Following continuous infusion of fluorescent macromolecules into pericardial space, concentrations in plasma are at least 7 fold lower than in pericardial fluid (Table 2). This is also the case for the small compounds cortisol and its 20-carbonic acid analogue (Table2). In contrast, following continuous infusion of macromolecules into blood, approximately similar concentrations were observed in pericardial fluid and in plasma.

Calculated clearances derived from steady-state concentrations (i.e. clearance=infusion dose rate/steady state concentration) in pericardial fluid upon intrapericardial infusion are 5.54±1.98 (FITC rat IgG), 4.23±0.75 (Texas Red RSA), 6.86±3.75 (Texas Red FGF-2), 3.91±0.31 μl/kg.min (FITC heparin) 105±29.3 (cortisol) and 30.8±3.52 μl/kg.min (cortisol carbonic acid). Calculated clearances from plasma steady state concentrations upon systemic infusion are 30.3±8.3 (FITC rat IgG), 54.2±18.4 (Texas Red RSA) and 36.1±3.64 μl/kg.min (Texas Red FGF-2). In some cases, these clearances are substantially lower than those calculated after bolus injection of the compounds (Table 1). This probably can be attributed to the existence of distribution processes that are saturated after long term infusion but not after bolus injection of the compounds, which results in an overestimation when calculating clearances for the bolus injections. Regarding FITC heparin, it should be kept in mind that the pharmacokinetics of heparins are known to be non-linear (Boneu et al., 1990), so that comparison between concentration profiles after bolus injections or infusions is difficult.

From these experiments it can be concluded that high drug concentrations in pericardial fluid can be obtained following intrapericardial application,, whereas plasma drug concentrations remain low. This can be explained by the fact that the clearances of substances in pericardial fluid are low, relative to substance clearances in plasma. Because of this pharmacokinetic advantage, a desirable local drug concentration may be achieved at lower doses, while the potential risk of peripheral side effects is reduced by intrapericardial drug application. Therefore, intrapericardial application of therapeutic agents provides a promising tool to obtain site-specific treatment of heart or coronary diseases.

### Example 7

Time Course of Infusion of Substances into the Pericardial Space

Substances were administered to the pericardial space of male Wistar rats weighing 250-300 grams by infusion via catheter for 1 week using an Alzet™ osmotic minipump at a volume rate of about 1 μl/hour. Blood and pericardial fluid samples were taken at various time points and the concentration of administered substances was measured fluorimetrically (for fluorescently labeled compounds) or by HPLC (for steroids). Concentration of fluorescently labeled compounds is expressed as fluorescent units/ml fluid.

Results

Albumin

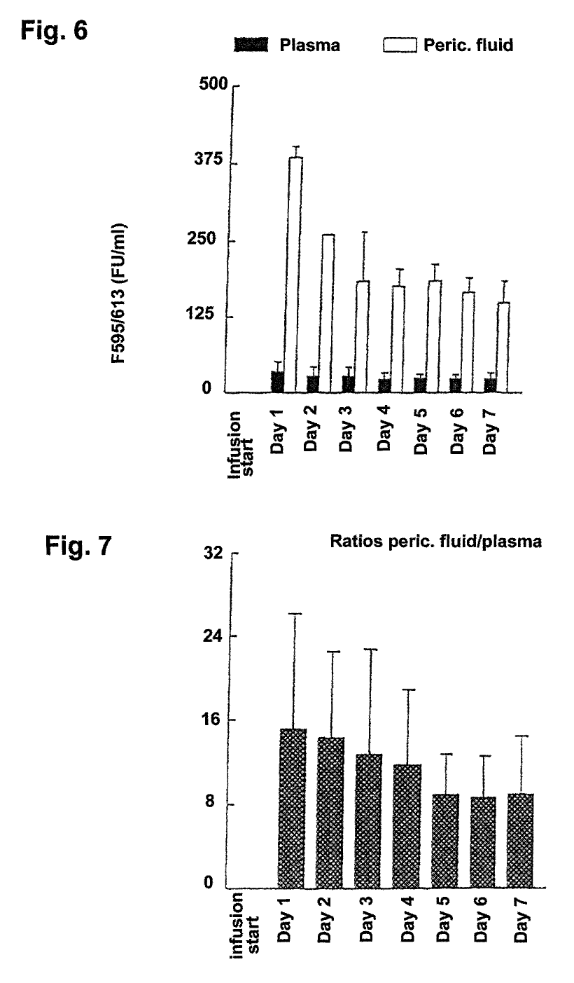

Texas red-labeled rat albumin was infused into the pericardial space and the concentration of labeled albumin in the pericardial fluid and in plasma was measured over time. The results are shown in FIG. 6. The plasma concentration (solid bars) of labeled albumin remained at a constant, low level over the 7-day period. The concentration of albumin in the pericardial fluid (open bars) dropped initially from about 375 FU/ml at day 1 after the start of infusion to about 190 FU/ml at day 3, and remained at this level through day 7.

As shown in FIG. 7, the ratio of the concentration of albumin in the pericardial fluid to the concentration in plasma ranged from about 9 to about 15 over the 7-day infusion period.

bFGF

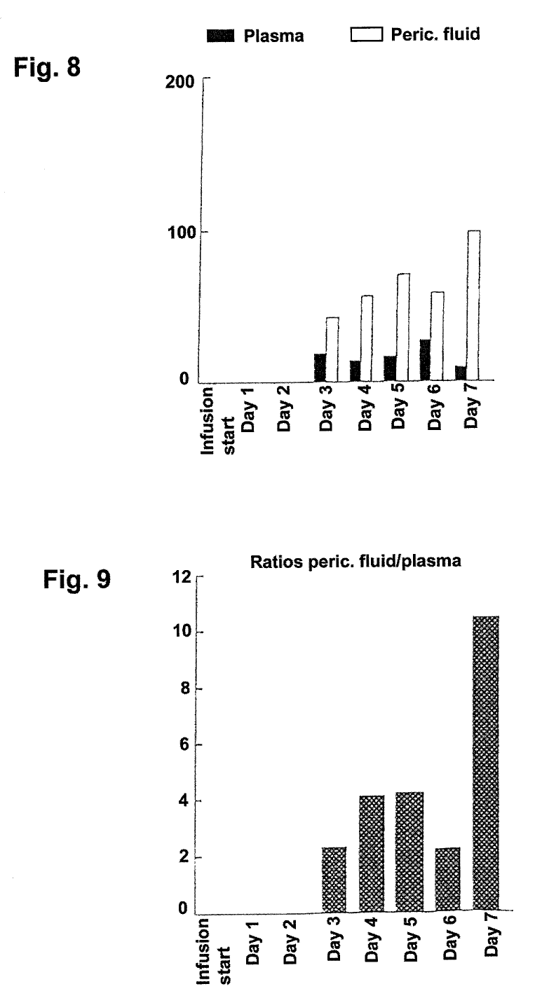

Texas red-labeled bFGF was infused into the pericardial space and the concentration of labeled bFGF in the pericardial fluid and in plasma was measured over time. The results are shown in FIG. 8. The plasma concentration (solid bars) of labeled bFGF remained at a low level from day 3 through day 7 after the start of infusion. The concentration of bFGF in the pericardial fluid (open bars) rose gradually between day 3 and day 7 after the start of infusion.

As shown in FIG. 9, the ratio of the concentration of bFGF in the pericardial fluid to the concentration in plasma ranged from about 2 to about 10 over days 3 to 7 of the 7-day infusion period.

Cortisol

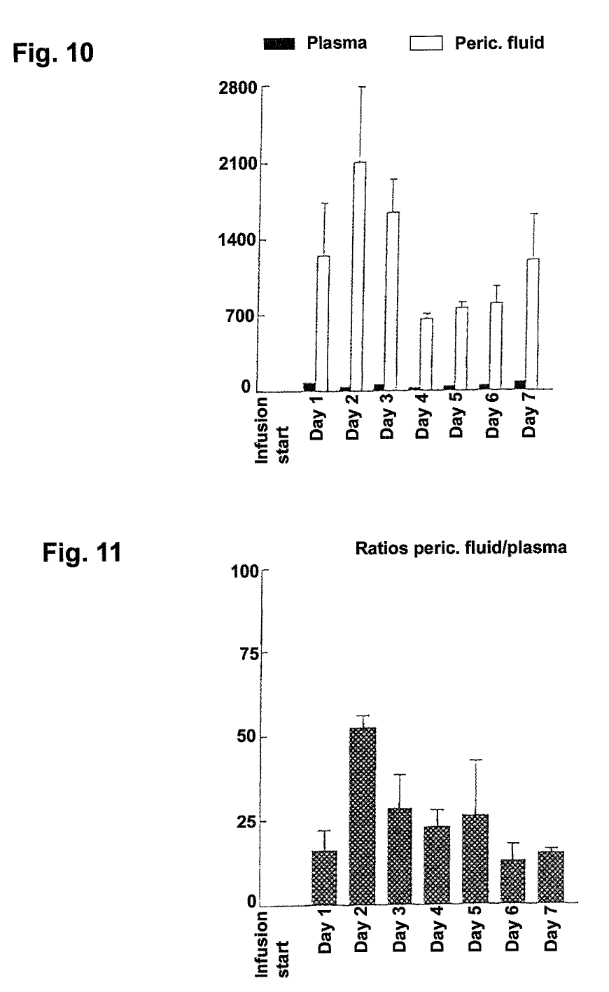

Cortisol was infused into the pericardial space and the concentration of cortisol in the pericardial fluid and in plasma was measured over time. The results are shown in FIG. 10. The plasma concentration (solid bars) of cortisol remained at a constant, low level over the 7-day period. The concentration of cortisol in the pericardial fluid (open bars) was between about 1000 nM and 2100 nM for the first three days of infusion, after which the concentration dropped, ranging from about 700 nM to about 1200 nm.

As shown in FIG. 11, the ratio of the concentration of cortisol in the pericardial fluid to the concentration in plasma ranged from about 12 to about 52 over the 7-day infusion period. The above results are summarized in Table 3 below.

TABLE 3
Summary of Ratio of Concentration of 7 Days Intrapercardial Infusion

| | Ratio of concentration in pericardial fluid to |
|---|---|
| | concentration in plasma |
| albumin | 9-15 |
| BFGF | 2-10 |
| cortisol | 12-50 |

The results indicate that, using continuous infusion of the substance over an extended period of time, (1) relatively constant amounts of a substance can be maintained in the pericardial space; and (2) relatively high ratio of the pericardial fluid concentration to plasma concentration can be maintained.

### Example 8

Effects of Bolus Injection Versus Infusion of FGF2 on Cardiac Function in Rats

The following example is provided to support the conclusion that sustained release of angiogenic factors is more effective than bolus administration in promoting neovascularization of cardiac tissue.

Study Design

Group 1: SHR; Intrapericardial Bolus Injection

Six spontaneous hypertensive rats (SHR) were given intrapericardial (ipc) bolus injections of fibroblast growth factor-2 plus heparin (FGF-2/heparin). A control group of six SHR rats were given ipc bolus injections of a solution of 1% rat serum albumin (RSA) in phosphate buffered saline (PBS). The amount of FGF-2 in the bolus injection of FGF-2/heparin was 336 micrograms/kg and 11 micrograms/kg body weight.

Group 2: SHR; Intrapericardial Infusion

Ten SHR rats were given FGF-2/heparin at 1000 ng/kg per hour or 33 ng/kg per hour for 14 days by ipc infusion. A control group of ten SHR rats were given RSA (1% in PBS) for 14 days by ipc infusion.

Group 3: SHR; Intravenous Infusion

Seven SHR rats were given FGF-2/heparin at 1000 ng/kg per hour or 33 ng/kg per hour for 14 days by intravenous (iv) infusion. A control group of eight SHR rats were given RSA (1% in PBS) for 14 days by iv infusion.

Group 4: WKY and SHR; No Treatment

Nine SHR rats served as untreated controls. Eight Wistar Kyoto (WKY; a strain of Rattus norvegicus used as normotensive controls for the SHR rat) were untreated and served as normotensive controls.

At day 0, catheters were implanted. At day 2, infusion began. At day 16, rats were sacrificed. Body weights and heart weights were determined. Capillary density was measured by staining cardiac sections with Griffonia simplicifolia lectin, and capillary:proprano ratios were determined with a combination of Griffonia simplicifolia lectin and a stain for laminin. Coronary blood flow (conductance) was determined on hearts ex vivo using retrograde Langendorff perfusion in the presence of nitroprusside/adenosine.

Results

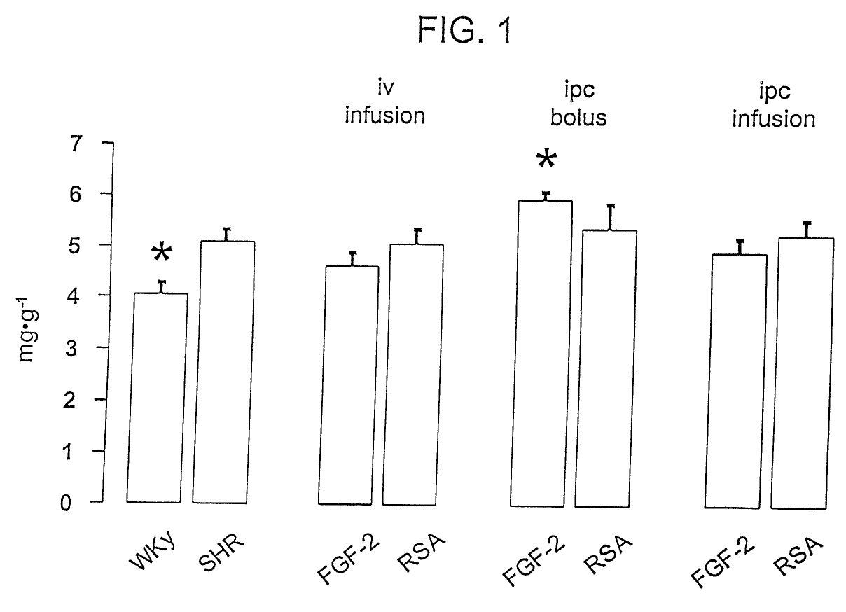

FIG. 1 shows the heart weight per body weight for the four groups of rats. As expected, untreated SHR rats' heart weights exceeded those of control WKY rats. Surprisingly, ipc bolus injection of FGF-2/heparin resulted in cardiac hypertrophy in SHR rats, such that the heart weight per body weight exceeded that of untreated SHR rats. Neither ipc nor iv infusion of FGF-2/heparin resulted in an increase in heart weight in SHR rats.

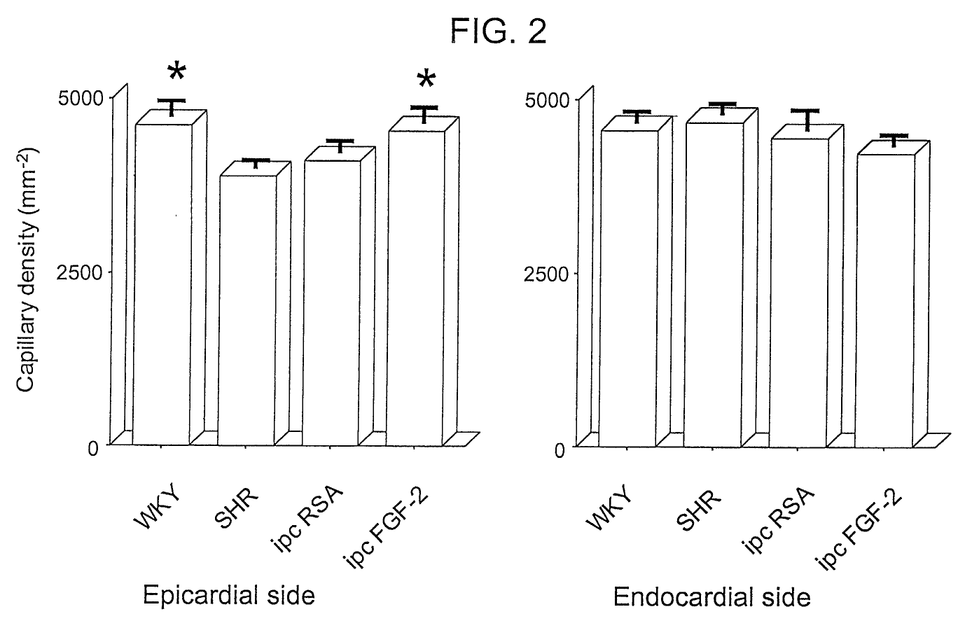

As shown in FIG. 2, cardiac capillary density (expressed as the number of capillaries per mm² of cardiac tissue) increased on the epicardial side, but not on the endocardial side, of SHR rats treated with FGF-2/heparin by ipc infusion.

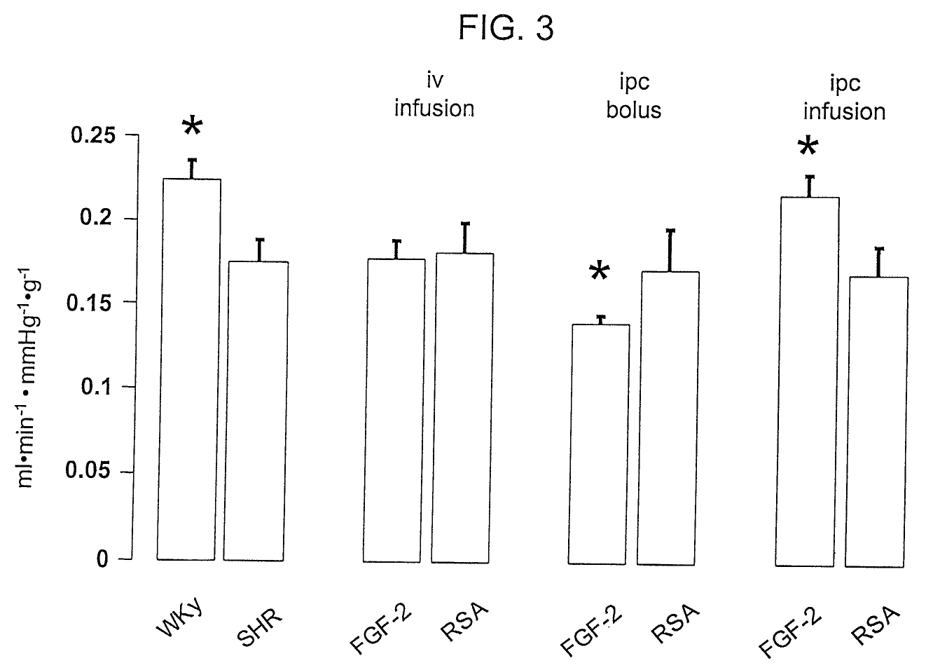

To determine whether the observed increase in capillary density resulted in increased blood flow in the heart (i.e., increased cardiac function), retrograde Langendorff perfusion was carried out on hearts ex vivo in the presence of nitroprusside/adenosine. The results are shown in FIG. 3. As expected, conductance, expressed as ml blood flow through the heart/(minute)(mmHg)(g), is significantly higher in control WKY rats than in untreated SHR rats. Intravenous infusion of FGF-2/heparin did not increase blood flow above untreated SHR levels. Intrapericardial bolus injection of FGF-2/heparin resulted in lower blood flow than untreated SHR levels. In contrast, ipc infusion of FGF-2/heparin resulted in increased blood flow, up to WKY control levels.

The results presented in Example 6 above demonstrate that the instant invention provides methods of increasing cardiac function. The results show that intrapericardial infusion of an angiogenic factor to the heart does not result in cardiac hypertrophy, increases capillary density, and restores coronary conductance (blood flow) to normal levels. In contrast, intravenous infusion of an angiogenic factor does not provide these positive effects. Furthermore, bolus injection of an angiogenic factor increases heart weight and reduces coronary conductance.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

References

Amano J, Suzuki A, Sunamori M, Shichiri M and Marumo F (1993) Atrial natriuretic peptide in the pericardial fluid of patients with heart disease. Clin. Sci. 85: 165-168.

Aoki M, Morishita R, Muraishi A, Moriguchi A, Sugimoto T, Maeda K, Dzay V J, Kaneda Y, Higaki J and Ogihara T (1997) Efficient in vivo gene transfer into the heart myocardial infarction model using the HVJ (hemagglutinating virus of Japan)—liposome method. J. Mol. Cell. Cardiol. 29: 949-959.

Ayers G M, Rho T H, Ben-David J, Besch H R and Zipes D P (1996) Amiodarone instilled into the canine pericardial sac migrates transmurally to produce electrophysiologic effects and suppress atrial fibrillation. J. Cardiovasc. Electrophysiol. 7: 713-721.

Boneu B, Caranobe C and Sie P (1990) Pharmacokinetics of heparin and low molecular weight heparin. Baillieres Clin. Haematol. 3: 531-544.

Buselmeier T J, Davin T D, Simmons R L, Najarian J S and Kjellstrand C M (1978) Treatment of intractable uremic pericardial effusion. Avoidance of pericardiectomy with local steroid instillation. JAMA 420: 1358-1359.

Corda S. Mebazaa A, Gandolfini M -P, Fitting C, Marotte F, Peynet J, Charlemagne D, Cavaillon J -M, Payen D, Rappaport L and Samuel J -L (1997) Trophic effect of human pericardial fluid on adult cardiac myocytes. Differential role of fibroblast growth factor-2 and factors related to ventricular hypertrophy. Circ. Res. 81: 679-687.

Darsinos J T, Karli J N, Samouilidou E C, Krumbholz B, Pistevos A C and Levis G M (1999) Distribution of amiodarone in heart tissues following intrapericardial administration. Int. J. Clin. Pharmacol. Ther. 37: 301-306.

Daemen M J A P, Smits J F M, Thijssen H H W and Struijker Boudier H A J (1988) Pharmacokinetic considerations in target-organ directed rug delivery. Trends Pharmacol. Sci 9: 138-141.

Eid H, Kuroski de Bold M L, Chen J H and de Bold A J (1994) Epicardial mesothelial cells synthesize and release endothelin. J. Cardiovasc. Pharmacol. 26: 715-720.

Gibson A T and Segal M B (1978) A study of the composition of pericardial fluid, with special reference to the probable mechanism of fluid formation. J. Physiol. 277: 367-377.

Horkay F, Laine M, Szokodi I, Leppätluoto J, Vuolteenaho O, Ruskoaho H, Juhász-Nagy A and Toth M (1995) Human pericardial fluid contains the highest amount of endothelin-1 of all mammalian biological fluids thus far tested. J. Cardiovasc. Pharmacol. 26 (Suppl3): S502-S504.

Klemola R, Laine M, Weckström M, Vuolteenaho O, Ruskoaho H, Huttunen O and Leppäluoto J (1995) High concentrations of atrial natriuretic peptide and brain natriuretic peptide in rat pericardial fluid and their reduction by reserpine in vivo. Naunyn-Schmiedeberg's Arch Pharmacol 352: 331-336.

Kleinjans J C, Smits J F M, van Essen H, Kasbergen C M and Struijker-Boudier H A J (1984) Hemodynamic characterization of hypertension induced by chronic intrarenal or intravenous infusion of norepinephrine in conscious rats. Hypertension 6: 689-699.

Kohnoe S, Maehara Y, Takahashi I, Saito A, Okada Y and Sugimachi K (1994) Intrapericardial mitomycin C for the management of malignant pericardial effusion secondary to gastric cancer. Chemotherapy 40: 57-60.

Kornowski R, Fuchs S, Leon M B and Epstein S E (2000) Delivery strategies to achieve therapeutic myocardial angiogenesis. Circulation 101: 454-458.

Labhasetwar V, Underwood T, Gallagher M, Murphy G, Langberg J and Levy R J (1994) Sotalol controlled-release systems for arrhythmias: in vitro characterization, in vivo drug disposition, and electrophysiologic effects. J. Pharm. Sci. 83: 156-164.

Laham R J, Rezaee M, Post M, Novicki D, Sellke F W, Pearlman J D, Simons M and Hung D (2000) Intrapericardial delivery of fibroblast growth factor-2 induces neovascularization in a porcine model of chronic myocardial ischemia. J. Pharmacol. Exp. Ther. 292: 795-802.

Landau C, Jacobs A K and Haudenschildt C C (1995) Intrapericardial basic fibroblast growth factor induces myocardial angiogenesis in a rabbit model of chronic ischemia. Am. Heart J. 129: 924-931.

Lazarous D F, Shou M, Stiber J A, Dadhania D M, Thirumurti V, Hodge E, and Unger E F (1997) Pharmacodynamics of basic fibroblast growth factor: route of administration determines myocardial and systemic distribution. Cardiovasc. Res. 36: 78-85.

Lazarous D F, Shou M, Stiber J A, Hodge E, Thirumurti V, Gonçalves L and Unger E F (1999).

Adenoviral-mediated gene transfer induces sustained pericardial VEGF expression in dogs: effect on myocardial angiogenesis. Cardiovasc. Res. 44: 294-302.

Lee H B and Blaufox M D (1985) Blood volume in the rat. J. Nucl. Med. 25: 72-76.

March K L, Woody M, Mehdi K, Zipes D P, Brantly M and Trapnell B C (1999) Efficient in vivo catheter-based pericardial gene transfer mediated by adenoviral vectors. Clin. Cardiol. 22 (1 Suppl 1): I23-I29.

Mason S R, Ward L C and Reilly P E B (1992) Fluorimetric detection of serum corticosterone using high-performance liquid chromatography. J. Chromatogr. 581: 267-271.

McDermott D A, Meller T M, Gebhart G F and Guttermann D D (1995) Use of an indwelling catheter for examining cardiovascular responses to pericardial administration of bradykinin in rat. Cardiovasc. Res. 30: 39-46.

Mebazaa A, Wetzel R C, Dodd-o J M, Redmond E M, Shah A M, Maeda K, Maistre G, Lakatta E G and Robotham J L (1998) Potential paracrine role of the pericardium in the regulation of cardiac function. Cardiovasc. Res. 40: 332-342.

Page E, Upshaw-Early J and Goings G (1992) Permeability of rat atrial endocardium, epicardium and myocardium to large molecules. Stretch-dependent effects. Circulation 71: 159-173.

Santamore W P, Constantinescu M S, Bogen D and Johnston W E (1990) Nonuniform distribution of normal pericardial fluid. Basic Res. Cardiol. 85: 541-549.

Sellke F W and Simons M (1999) Angiogenesis in cardiovascular disease. Current status and therapeutic potential. Drugs 58: 391-396.

Smits J F M, Coleman T G, Smith T L, Kasbergen C M, van Essen H and Struijker-Boudier H A J (1982) Antihypertensive effect of ropranolol in conscious spontaneously hypertensive rats: central hemodynamics, plasma volume and renal function during β-blockade with ropranolol. J. Cardiovasc. Pharmacol. 4: 904-914.

Smits J F M and Thijssen H H W (1986) Spatial control of drug action: theoretical considerations on the pharmacokinetics of target-aimed drug delivery, in Rate-controlled drug administration and action (Struijker Boudier H A J, ed) pp 83-114, CRC Press, Boca Raton, Fla.

Spodick D H (1992) Macrophysiology, microphysiology and anatomy of the pericardium: a synopsis. Am. Heart. J. 124: 1046-1051.

Spodick D H (2000) Intrapericardial therapeutics and diagnostics. Am. J. Cardiol. 85: 1012-1014.

Stoll H P, Szabo A and March K L (1998) Sustained transmyocardial loading with bFGF following single intrapericardial delivery: local kinetics and tissue penetration. Circulation 98:1-2100.

Uchida Y, Yanagisawa-Miwa A, Nakamura F, Yamada K, Tomaru T, Kimura K and Morita T (1995) Angiogenic therapy of acute myocardial infarction by intrapericardial injection of basic fibroblast growth factor and heparin sulfate: an experimental study. Am. Heart J. 130: 1182-1188.

Veelken R, Sawin L L and Dibona G F (1990) Epicardial serotonin receptors in circulatory control in conscious Sprague-Dawley rats. Am. J. Physiol 258: H466-H472.

Vogel A I (1956) A text-book of practical organic chemistry including qualitative organic analysis. Third edition. Longman Group Ltd, London, UK.

Wauquier I and Devynck M A (1989) Body fluid variations and endogenous digitalis-like compounds during chronic NaCl loading in Wistar rats. Clin. Exp. Hypert. A11: 1217-1234.

Waxman S, Moreno R, Rowe K A and Verrier R L (1999) Persistent primary coronary dilation induced by transatrial delivery of nitroglycerin into the pericardial space: a novel approach for local cardiac drug delivery. J. Am. Coll. Cardiol. 33: 2073-2077.

Willerson J T, Igo S R, Sheng-Kun Y, Ober J C, Macris M P and Ferguson J J (1996) Localized administration of sodium nitroprusside enhances its protection against platelet aggregation in stenosed and injured coronary arteries. Texas Heart Inst. J. 23: 1-8.

Zhang J C L, Woo Y J, Chen J -A, Swain J L and Sweeney (1999) Efficient transmural cardiac gene transfer by intrapericardial injection in neonatal mice. J. Mol. Cell. Cardiol. 31: 721-732.

1. A method for improving cardiac function in a subject, the method comprising: implanting in said subject a sustained release dosage form, said sustained release dosage form comprising a drug delivery device and a cardiac drug, and administering said cardiac drug from said dosage form into said subject, for a period of at least 24 hours, in an dose sufficient to cause a measurable improvement in cardiac function.

2. The method of claim 1, wherein said dosage form is implanted within the pericardium or myocardial tissue or cardiac vasculature of said subject.

3. The method of claim 2, wherein said drug delivery device is selected from the group consisting of: a pump, a bioerodable implant, and a depot.

4. The method of claim 3, wherein said cardiac drug is selected from the group consisting of: an angiogenic factor, growth factor, calcium channel blocker, antihypertensive agent, inotropic agent, antiatherogenic agent, anti-coagulant, beta-blocker, anti-arrhythmic agent, anti-inflammatory agent, sympathomimetic agent, phosphodiesterase inhibitor, diuretic, vasodilator, thrombolytic agent, cardiac glycoside, antibiotic, antiviral agent, antifungal agent, antineoplastic agent, and steroid.

5. The method of claim 4, wherein said cardiac drug is an angiogenic factor.

6. The method of claim 4, wherein said dosage form comprises a depot.

7. The method of claim 6, wherein said depot comprises a non-polymeric high viscosity material having a viscosity of at least 5000 cP at 37° C.

8. The method of claim 7, wherein said high viscosity material comprises sucrose acetate isobutyrate.

9. The method of claim 4, wherein said dosage form comprises a biodegradable implant.

10. The method of claim 9, wherein said biodegradable implant comprises a biodegradable polymer.

11. The method of claim 10, wherein said polymer comprises poly (DL-lactide-co-glycolide).

12. The method of claim 2, wherein said drug delivery device comprises a microsphere formulation, and wherein said cardiac drug is selected from the group consisting of: an angiogenic factor, growth factor, calcium channel blocker, antihypertensive agent, inotropic agent, antiatherogenic agent, anti-coagulant, beta-blocker, anti-arrhythmic agent, anti-inflammatory agent, sympathomimetic agent, phosphodiesterase inhibitor, diuretic, vasodilator, thrombolytic agent, cardiac glycoside, antibiotic, antiviral agent, antifungal agent, antineoplastic agent, and steroid.

13. The method of claim 12, wherein said microsphere formulation comprises a polymer selected from the group consisting of poly(DL-lactide-co-glycolide), polycaprolactone, polyglycolide, and combinations thereof.

14. The method of claim 13, wherein said microsphere formulation comprises an angiogenic factor.

15. The method of claim 4, wherein said dosage form comprises a pump.

16. The method of claim 15, wherein said pump is operatively attached to a catheter.

17. The method of claim 16, wherein said pump is implanted outside the pericardial space, and wherein said catheter delivers said cardiac drug from said pump to said myocardial tissue.

18. A method for promoting angiogenesis in the heart or cardiac vasculature a subject, the method comprising: implanting in the heart or cardiac vasculature of said subject a sustained release dosage form, said sustained release dosage form comprising a non-polymeric depot, and an angiogenic factor, and administering said angiogenic factor from said non-polymeric depot into said subject, for a period of at least 24 hours, in an dose sufficient to cause a measurable angiogenesis in the heart or cardiac vasculature of said subject.

19. An implantable dosage form comprising a drug delivery device and a cardiac drug wherein said drug delivery device is selected from the group consisting of: a bioerodable implant, a depot, and a microsphere formulation, and wherein said cardiac drug is selected from the group consisting of: an angiogenic factor, growth factor, calcium channel blocker, antihypertensive agent, inotropic agent, antiatherogenic agent, anti-coagulant, beta-blocker, anti-arrhythmic agent, anti-inflammatory agent, sympathomimetic agent, phosphodiesterase inhibitor, diuretic, vasodilator, thrombolytic agent, cardiac glycoside, antibiotic, antiviral agent, antifungal agent, antineoplastic agent, and a steroid.

20. The implantable dosage form of claim 19 wherein the drug delivery device comprises a non-polymeric high viscosity material having a viscosity of at least 5000 cP at 37° C.

21. The method of claim 20, wherein said high viscosity material comprises sucrose acetate isobutyrate.

22. The method of claim 19, wherein said dosage form comprises a bioerodable implant.

23. The method of claim 19, wherein said drug delivery device comprises a microsphere formulation.

24. The method of claim 4, wherein the cardiac drug is an angiogenic factor and wherein said angiogenic factor is selected from the group consisting of a basic fibroblast growth factor, an acidic fibroblast growth factor, a vascular endothelial cell growth factor, transforming growth factor-α, transforming growth factor-β, platelet derived growth factor, an endothelial mitogenic growth factor, platelet activating factor, tumor necrosis factor-α, angiogenin, a prostaglandin, placental growth factor, granulocyte colony stimulating factor, hepatocyte growth factor, interleukin-8, vascular permeability factor, epidermal growth factor, substance P, bradykinin, angiogenin, angiotensin II, proliferin, insulin like growth factor-1, nicotinamide, a stimulator of nitric oxide synthase, and estrogen.

25. The method of claim 24, wherein the drug is delivered at a volume rate of from about 0.01 μl/day to about 2 ml/day.

26. The method of claim 25, wherein said administering is for a period of from about 2 weeks to about 12 months.

27. The method of claim 26, wherein the controlled release drug delivery device comprises a depot.

28. The method of claim 27, wherein the depot comprises sucrose acetate isobutyrate.

29. An implantable sustained release dosage form for improving cardiac function in a subject, the dosage form comprising a drug delivery device and a cardiac drug, wherein said drug delivery device contains sufficient drug to allow administration of said cardiac drug to the subject for a period of at least 24 hours in a dose sufficient to cause a measurable improvement in cardiac function.

30. The device of claim 29 wherein said dosage form is implanted within the pericardium or myocardial tissue or cardiac vasculature of said subject.

31. The device of claim 30 wherein said drug delivery device is selected from the group consisting of: a pump, a bioerodable implant, and a depot.

32. The device of claim 31 wherein the cardiac drug is selected from the group consisting of: an angiogenic factor, and anti-arrhythmic agent, and antihypertensive agent and a steroid.

33. The device of claim 32 wherein the drug delivery device is a pump.

34. The device of claim 32 wherein the drug delivery device is a depot.

35. The device of claim 32 wherein the drug delivery device is a bioerodable implant.
